(19) United States
(12) Patent Application Publication
(10) Pub. No.: US 2026/0294390 A1
(43) Pub. Date: Oct. 1, 2026

(54) MEDICAL DEVICES WITH INTEGRATED ULTRASOUND OPTICAL SENSORS

(71) Applicant: DEEPSIGHT TECHNOLOGY, INC., SANTA CLARA, CA (US)

(72) Inventors: Michael Hazarian, San Jose, CA (US); Joshua Arnone, St. Peters, MO (US); Yingyi Zhang, Fremont, CA (US); Robert Steins, Sausalito, CA (US); Sitai Kou, Brentwood, MO (US); Linhua Xu, UNIVERSITY CITY, MO (US); Jiangang Zhu, St. Louis, MO (US); Lan Yang, Clayton, MO (US); Bradford Wood, Santa Clara, CA (US); Andrew Dove, St. Louis, MO (US); Thomas Horning, St. Louis, MO (US)

(73) Assignee: DEEPSIGHT TECHNOLOGY, INC., SANTA CLARA, CA (US)

(21) Appl. No.: 19/630,961

(22) Filed: Mar. 27, 2026

Related U.S. Application Data
(60) Provisional application No. 63/779,440, filed on Mar. 28, 2025.
(60) Provisional application No. 63/779,812, filed on Mar. 28, 2025.
(60) Provisional application No. 63/942,998, filed on Dec. 17, 2025.

(51) Int. Cl. A61B 8/12; A61B 8/00

(52) U.S. Cl. CPC A61B 8/12; A61B 8/42; A61B 8/5207

(57) ABSTRACT

The medical device with an integrated ultrasound optical sensor includes a device proximal end, a body extending from the device proximal end, and at least one ultrasound sensor secured to the body. The at least one ultrasound sensor may be an optical ultrasound sensor with a sensor portion and an optical fiber in optical communication therewith. The sensor portion is positioned adjacent a distal end of the body of the medical device. A chip-based laser may be secured to the device proximal end, with the at least one ultrasound sensor being in communication with the chip-based laser.

### CROSS-REFERENCE TO RELATED APPLICATIONS

This application claims the benefit of U.S. Provisional Patent Application No. 63/779,440, filed on Mar. 28, 2025, and also claims the benefit of U.S. Provisional Application No. 63/942,998, filed on Dec. 17, 2025, and further claims the benefit of U.S. Provisional Application No. 63/779,812, filed on Mar. 28, 2025, each of which is hereby incorporated by reference in its entirety.

### BACKGROUND

### Field

The disclosure of the present patent application relates to medical devices and instruments, and particularly to medical devices and instruments with integrated ultrasound optical sensors.

### Description of Related Art

Ultrasound imaging is used in medical procedures to visualize and provide internal imaging of a patient's body, often with a view from the outside (transcutaneous) or a view from the inside (endoscopic, endobronchial, intravascular, or laparoscopic). Ultrasound imaging is also used to plan, localize, visualize and track medical tools and devices during procedures and placement of devices, such as needles, introducers, trocars, delivery sheaths, dilators, catheters, guidewires, endoscopes, tubes (e.g., endotracheal tubes, nasogastric (NG) tubes, gastric tubes, nephrostomy tubes, etc.), and the like. Ultrasound may also be used to confirm device location, therapeutic bioeffects, or assess for complications. Ultrasound imaging is a non-invasive form of imaging with a relatively high degree of penetration depth within tissues, but not infinite, and imaging quality and ability reduces with depth and body mass index (BMI).

Some conventional existing imaging technologies use Acoustic Energy Generating (AEG) materials for transducers to visualize and track medical objects and to generate imagery during a diagnostic or therapeutic medical procedure. Commonly used AEG materials include piezoelectric materials such as lead-zirconate-titanate (PZT), ceramic, piezoelectric single crystal (e.g. PIN-PT, PIN-PMN-PT), and polyvinylidene fluoride (PVDF) among many other materials known to those of skill in the art. AEG transducers have limitations. The echogenicity of the object to be tracked and/or anatomy being visualized can affect the image quality of the object being tracked and the tissue being imaged. In certain medical procedures a small form factor is needed, and small AEG transducers generally have low to minimal signal output. Therefore, it may be challenging to use AEG transducers for medical applications requiring a small form factor because of the size limitations (e.g., physical size). Thus, medical devices with integrated ultrasound optical sensors solving the aforementioned problems are desired.

### SUMMARY

The medical device with an integrated ultrasound optical sensor includes a device proximal end, such as a hub, luer lock, port, connector, handle or the like, a body extending from the device proximal hub, and at least one ultrasound sensor secured to the body. The at least one ultrasound sensor may be an acousto-optic ultrasound sensor with a sensor portion and an optical fiber in optical communication therewith. The sensor portion is positioned adjacent a distal end of the body of the medical device, or other clinically relevant locations, such as fixed distances along the shaft or delivery vehicle, such as proximal to the tip, or at expected or potential margins of therapeutic effects (thermal ablation, electroporation, histotripsy, or drug or hydrogel delivery). A chip-based laser may be secured to the device proximal end, with the at least one ultrasound sensor being in communication with the chip-based laser. Alternatively, the ultrasound sensor may be in communication with a non-chip based laser coupled to the medical device.

The medical device with an integrated ultrasound optical sensor may include a device proximal end, a body extending from the device proximal end, a device distal end, a working section on the body, at least one ultrasound optical sensor secured to the body proximate the distal end, a light source configured to provide light to the optical ultrasound sensor via the optical fiber, a photodetector configured to receive an optical signal from the optical ultrasound sensor, and a controller. The controller may be configured to control the light source, receive optical data based on the optical signal from the photodetector, and perform at least one of image generation and location determination based on the optical data.

The at least one ultrasound optical sensor may be calibrated to provide multimodality data of temperature, interstitial pressure or other biological characteristics. The controller may be further configured to receive the multimodality data from the at least one ultrasound optical sensor, process the multimodality data using a machine learning model, and generate at least one output including an ultrasound-centered model for determining a finite analysis of tissue sensed by the at least one ultrasound optical sensor to determine Pennes Bioheat equation effects upon the tissue, an indication of a cumulative lethal tissue zone, an ultrasound-centered model with spatial coordinates for interstitial pressure, or co-register point-based pressure measurement spatial data to match sensor data with a magnetic resonance imaging (MRI) diffusion-weighted imaging/apparent diffusion coefficient (DWI/ADC) map.

In an embodiment, the body may have at least one groove formed in an outer surface thereof, with the at least one ultrasound sensor being received within the at least one groove. As a non-limiting example, the at least one groove may be a helical groove. The at least one groove and the at least one ultrasound sensor may be covered with a conformal coating, such as a protective coating or a protective sheath.

In another embodiment, the at least one ultrasound sensor may be wrapped around the body. The at least one ultrasound sensor may be covered with protective coating(s) or protective sheath(s).

In another embodiment, at least one lumen is provided. The at least one ultrasound sensor may be slidably received within the at least one lumen.

In another embodiment, the body may be hollow, with the at least one ultrasound sensor received within the body. The distal end of the body may have at least one window formed therein, with the sensor portion of the at least one ultrasound sensor positioned within the at least one window. The sensor fiber may further include fiber Bragg gratings proximal to a distal acousto-optic sensor, thus creating hybrid sensors with FBG in-line sensors and an acousto-optic sensor at the fiber-end.

In another embodiment, the body is hollow and the at least one ultrasound sensor is secured to the body. The body may be covered with a cover layer, with the optical fiber embedded therein. An annular layer may be positioned adjacent the cover layer, with the sensor portion embedded in the annular layer. The annular layer may be formed from an acoustic matching material.

In an embodiment, the optical sensor is an acousto-optic sensor that can be configured/optimized for in situ tracking, imaging and/or environmental or physiologic sensing (e.g., pressure, temperature, nerve activity), with the medical device being used in interventional procedures, and may be a guidewire. Because the guidewire may act as a working wire over which instruments are placed for stent graft deployment or sizing, balloon deployment, recanalization or crossing device, thrombectomy or other interventions, the proximal end includes a selectively removable/detachable hub to disconnect the sensor from the laser light source, permit the instruments such as guide catheters, balloon catheters and the like to be introduced over the guidewire, and then allow “reconnection” of the fiber sensor to the laser to continue permitting imaging by the distally positioned acousto-optic sensor or other in situ environmental or physiologic sensing such as temperature, pressure or nerve activity.

In another embodiment, a fiber or guidewire harness/clip that enables device advancement over a wire with subsequent reconnection for in situ imaging or environmental or physiologic sensing is located at the proximal end.

A working guidewire with an integrated ultrasound optical sensor is further provided, including a guidewire having opposed distal and proximal ends, with an acoustic window formed in the distal end. An acousto-optic sensor is received within the guidewire, with a fiber-end sensor portion thereof positioned within the acoustic window. An additional sensor may also be received within the guidewire to form a hybrid sensor system. The additional sensor may include, as a non-limiting example, an optical fiber with at least one fiber Bragg grating (FBG). The additional sensor may be, as another non-limiting example, an extrinsic Fabry-Pérot interferometer (EFPI) for making pressure measurements.

In another embodiment, a sensorized introducer is provided, including an introducer tube having opposed distal and proximal ends. A surface of the introducer tube has a groove formed therein. The sensorized introducer further includes an introducer hub, with the proximal end of the introducer being received by the introducer hub. An acousto-optic sensor is received within the groove, with a fiber-end sensor portion thereof positioned adjacent the distal end of the introducer tube. A proximal portion of an optical fiber of the acousto-optic sensor extends through the introducer hub. The fiber-end sensor portion and at least a portion of the optical fiber may be bonded within the groove, and the introducer tube may be coated with a conformal or protective coating. A fiber mount may be mounted on the introducer hub for carrying at least a portion of the optical fiber.

Each of the above embodiments may further include a light source configured to provide light to the acousto-optic sensor via the optical fiber, a photodetector configured to receive an optical signal from the optical ultrasound sensor, and a controller. The controller may be any suitable type of controller, such as, but not limited to, a processor, a computer, a programmable logic controller, control circuitry or the like. The controller is configured to control the light source, receive optical data based on the optical signal from the photodetector, and perform at least one of image generation and location determination based on the optical data in addition to environmental or physiologic sensing.

In a further embodiment, a robotic assisted surgical system includes at least one end effector, and the medical device with an integrated ultrasound optical sensor coupled to the at least one end effector.

These and other features of the present subject matter will become readily apparent upon further review of the following specification.

### BRIEF DESCRIPTION OF DRAWINGS

FIG. 1, FIG. 2A, FIG. 2C and FIG. 2E illustrate embodiments of a medical device with an integrated ultrasound optical sensor incorporating ultrasound sensors into a stylet.

FIG. 2B is a cross-sectional view taken along cross-sectional cut line 2-2 in FIG. 2A.

FIG. 2D is a blown-up portion of FIG. 2C, particularly the portion of FIG. 2C bounded by broken lines.

FIG. 3A illustrates an embodiment of a medical device with an integrated ultrasound optical sensor incorporating ultrasound sensors into guidewires.

FIG. 3B is a cross-sectional view taken along cross-sectional cut line 3-3 in FIG. 3A.

FIG. 3C is a blown-up portion of a distal body of the medical device of FIG. 3B, particularly the portion of FIG. 3B shown bounded by the distal broken line box.

FIG. 3D is a blown-up portion of a proximal body of the medical device of FIG. 3C, particularly the portion of FIG. 3B shown bounded by the proximal broken line box.

FIG. 3E is a partial cross-sectional view taken along cross-sectional cut line 3-3 in FIG. 3A.

FIG. 3F is a partial perspective view in section of the medical device of FIG. 3A.

FIG. 3G is a partial side view in section of the medical device of FIG. 3A.

FIG. 3H is a side view in section of a connector for the proximal body of the medical device of FIG. 3A.

FIG. 4A illustrates an embodiment of a medical device with an integrated ultrasound optical sensor incorporating ultrasound sensors into catheters.

FIG. 4B is a cross-sectional view taken along cross-sectional cut line 4-4 in FIG. 4A.

FIG. 4C diagrammatically illustrates the proximal end of the medical device terminating in an optical connector.

FIG. 4D diagrammatically illustrates the proximal end of the medical device terminating in a hybrid optical-electrical connector.

FIG. 4E diagrammatically illustrates an alternative embodiment of the medical device in wireless communication with a backend system.

FIG. 4F diagrammatically illustrates an embodiment of the medical device with a single sensor in a single lumen catheter.

FIG. 4G diagrammatically illustrates another embodiment of the medical device with multiple sensors in a single lumen catheter.

FIG. 4H diagrammatically illustrates another embodiment of the medical device with multiple sensors within multiple lumens in a catheter.

FIG. 5A, FIG. 5B and FIG. 5C illustrate an embodiment of the medical device with an integrated ultrasound optical sensor incorporating an ultrasound sensor into a tubular-shaped medical device. FIG. 5A is shown partially cut away.

FIG. 6 illustrates an embodiment of an ultrasound sensor incorporating multiple integrated sensors.

FIG. 7 illustrates an optical system which may be used in the medical device with an integrated ultrasound optical sensor.

FIG. 8A diagrammatically illustrates a system design for a hybrid optical fiber sensor.

FIG. 8B diagrammatically illustrates a distal end of the hybrid optical fiber sensor of FIG. 8A.

FIG. 9A diagrammatically illustrates an alternative system design for a hybrid optical fiber sensor.

FIG. 9B diagrammatically illustrates a distal end of the hybrid optical fiber sensor of FIG. 9A.

FIG. 10 illustrates an alternative embodiment of the hybrid fiber sensor incorporating an extrinsic Fabry-Pérot interferometer (EFPI).

FIG. 11A is an exploded environmental view of a proximal end fiber connector.

FIG. 11B is an exploded environmental view of an alternative proximal end fiber connector.

FIG. 11C diagrammatically illustrates another alternative proximal end fiber connector.

FIG. 11D, FIG. 11E, FIG. 11F, FIG. 11G and FIG. 11H illustrate further alternative proximal end fiber connectors.

FIG. 12A diagrammatically illustrates a fiber-end sensor integrated into the distal end of a working guidewire.

FIG. 12B diagrammatically illustrates an alternative fiber-end sensor integrated into the distal end of a working guidewire.

FIG. 12C diagrammatically illustrates another alternative fiber-end sensor integrated into the distal end of a working guidewire.

FIG. 13 illustrates an embodiment of 1.8 Fr sensorized catheter incorporating a fiber-end sensor (FES) and a transition region, which allows the coupling and decoupling of the fiber-end sensor to the laser source, thereby permitting coaxial introduction of a instrument over the guidewire.

FIG. 14A illustrates an embodiment of a sensorized introducer incorporating a fiber-end sensor.

FIG. 14B is a partial perspective view in section of the sensorized introducer of FIG. 14A.

FIG. 14C is a partial side view in section of the sensorized introducer of FIG. 14A.

FIG. 14D is a partial side view of the sensorized introducer of FIG. 14A.

FIG. 15 is a block diagram illustrating system components of a robotic assisted surgical system.

Similar reference characters denote corresponding features consistently throughout the attached drawings.

### DETAILED DESCRIPTION

Non-limiting examples of various aspects and variations of the invention are described herein and illustrated in the accompanying drawings. The following detailed description is merely illustrative in nature and is not intended to limit the invention or the application and uses of the invention. Although the description of the invention is in the context of optical sensor systems, methods, and devices for ultrasound imaging, the disclosure should not be considered so limiting. For example, although methods may be discussed herein with respect to medical ultrasound, embodiments hereof may be suitable for other medical procedures as well as other procedures or methods in other industries that may benefit from the sensing and imaging technologies described herein. Further, various systems and devices that incorporate optical sensors are described. It should be understood that optical sensors, as described herein, may be integrated into and/or used with a variety of systems and devices not described herein. Modifications may be made to the embodiments described herein without departing from the spirit and scope of the present invention. Therefore, the following detailed description is not meant to be limiting. Furthermore, there is no intention to be bound by any expressed or implied theory presented in the preceding technical field, background, summary, or the following detailed description.

Various structures are described herein according to their geometric properties. As discussed herein, all structures so described may vary from the described shape according to the tolerances of known manufacturing techniques. Unless otherwise specified, features described with the term “substantially” are understood to be within 5% of exactness. For example, features described as “substantially parallel” may deviate from true parallel by 5%.

Certain interventional procedures utilize ultrasound for visualization of the patient's anatomy before and/or during the procedure along with visualization, tracking and positioning of the medical instrument during the procedure. The present invention is directed to incorporating optical sensors on a guidewire, catheter or other interventional instrument, ranging from an introducer, fiducial or instrument stylet to elongated tools such as catheters, microcatheters, guidewires, fiducials and the like. Furthermore, the interventional instrument may also be designed to remain within the patient for monitoring purposes. Interventional instruments vary in the type of invasiveness and size, form factor, rigidity, bending and robustness of the instrument, insertion and positioning of the instrument (steerable or stress induced), mechanical motion control, sensor protection and functionality, as well as response time and sensitivity. Interventional instrumentation also varies in required signal readouts (e.g., acoustic signals, temperature signals, pressure signals, etc.), metrologics, and concerns regarding whether the instrument affect a sensed item, such as, for example, a catheter obstructing the blood flood and, thus, affecting the measurement. Interventional instruments also vary in their usage; i.e., disposable vs. reusable instruments.

The medical device with an integrated ultrasound optical sensor, or sensorized instrument, includes a device proximal end, such as a hub, luer lock, port, connector, handle or the like, a body extending from the device proximal hub, and an ultrasound sensor secured to the body at a desired location at, or approximately located at, the distal or working portion of the instrument. The ultrasound sensor may be an acousto-optical ultrasound sensor with a sensor portion and at least one optical fiber in optical communication therewith or other spectral windows, with the sensor portion positioned adjacent a distal end of the body. It should be understood that the ultrasound sensor may be any suitable type of optical ultrasound sensor, such as those described in U.S. Pat. No. 12,025,489 B1 and U.S. Patent Publication Nos. US 2022/0365036 A1, US 2023/0097639 A1, US 2022/0350022 A1, US 2023/0148869 A1, and US 2024/0426650 A1, as well as co-pending U.S. application Ser. No. 19/403,738, titled “Fiber Sensors for Interventional Tools,” filed on Nov. 28, 2025, each of which is hereby incorporated by reference.

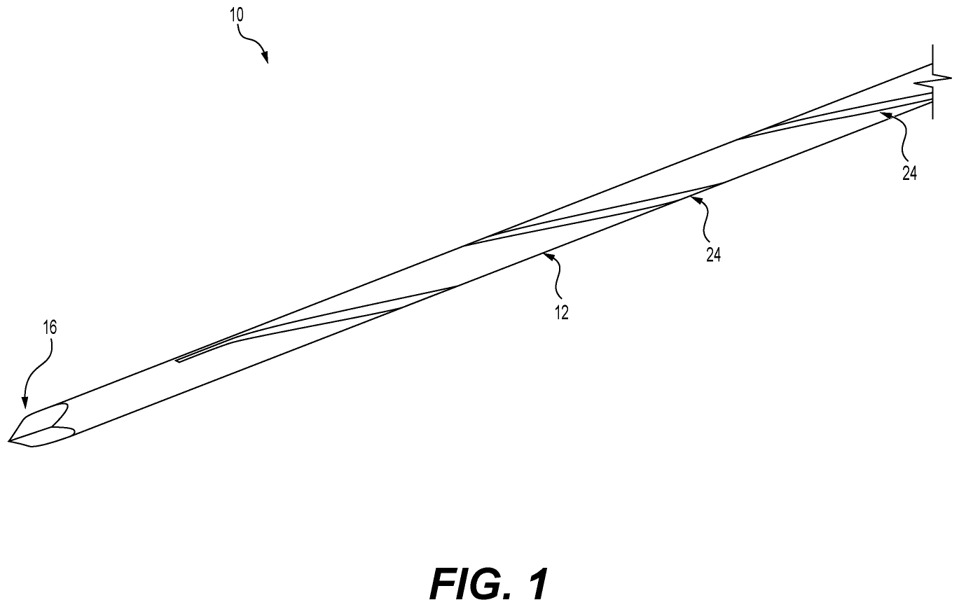

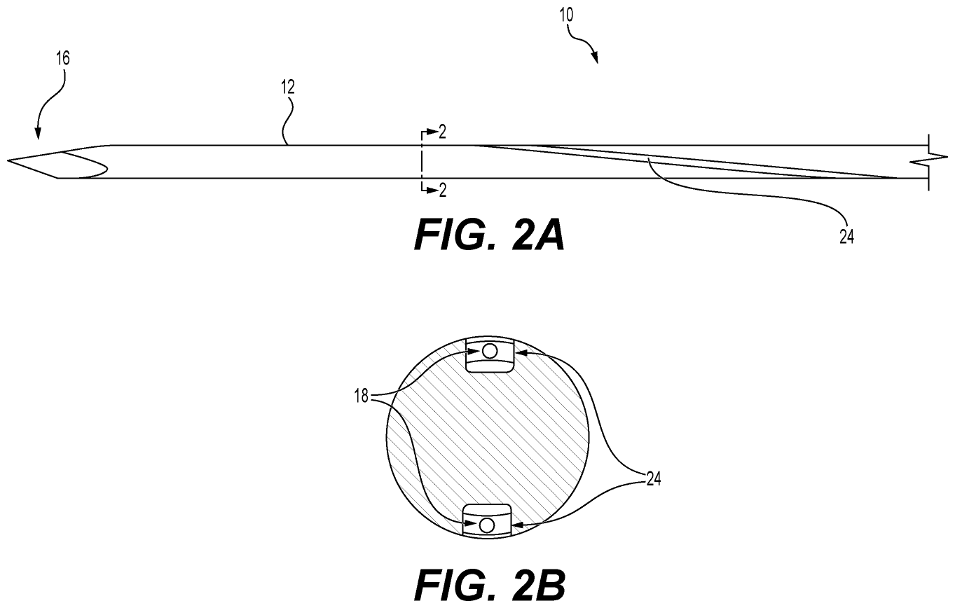

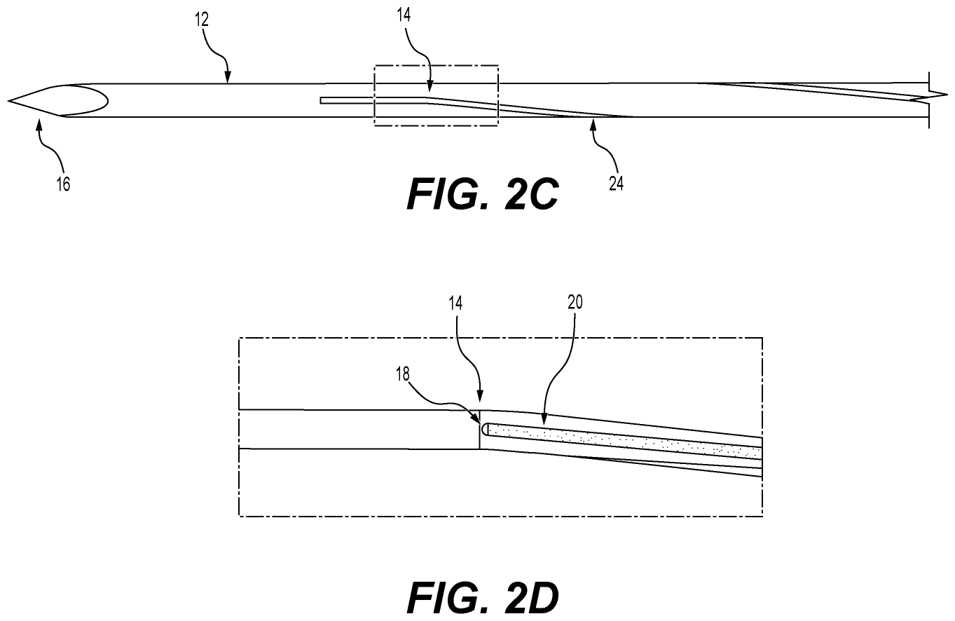

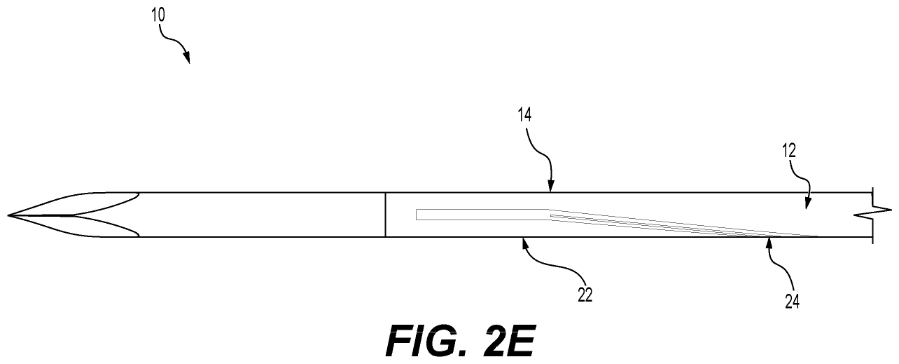

A chip-based laser (OSoC) or a fiber-based laser may be secured to the device proximal hub, or embedded therein, providing an on-device light source for the ultrasound sensor, which is in communication with the chip-based laser or fiber-based laser. The chip-based laser is particularly well-suited for sensors requiring multiple light sources simultaneously. In other embodiments, the sensorized instrument optical sensor may be tethered via optical cables to a laser located at a system controller housing a laser, optical/electronic components and data processing. In the non-limiting example of FIGS. 1 and 2A-2D, the elongated body 12 of an interventional instrument 10 is shown, with the sensor portion 14 positioned adjacent a tapered distal end 16. While the distal tip 16 is shown as being tapered, the actual geometry will depend upon the instrument. The interventional instrument 10 may be one for conducting procedures, or used during procedures, such as a stylet, an introducer, a fiducial, a biopsy instrument, an ablation instrument, a transcatheter procedure instrument (such as transcatheter aortic valve replacement (TAVR), transcatheter mitral valve replacement (TMVR), or transcatheter edge-to-edge repair (TEER)) or other minimally invasive/interventional procedures. It should be understood that the instrument 10, as shown, is a non-limiting example and is representative of the generally cylindrical instruments used in minimally invasive/interventional procedures. In the event of an ablation instrument, as a non-limiting example, such an ablation instrument may be a thermal ablation instrument, radio frequency (RF) ablation instrument, microwave ablation instrument or cryoablation instrument, and include various ablation instruments for tumor ablation, cardiac ablation, pain management and other procedures. The medical device may also be a sensorized instrument used to facilitate the interventional instrument or devices, such as, but not limited to, a biopsy instrument, a guide catheter, a catheter, a delivery catheter, drains, an introducer, a sheath, a probe, a fiducial or other instrument. It should be further understood that the sensorized instrument 10 may be rigid or flexible, and may be used during a procedure and/or used for patient monitoring prior, during and/or after a procedure. The inclusion of a sensor or sensors 18 on indwelling tubes or catheters will enable monitoring scenarios, such as the monitoring of endotracheal tubes (lung and cardiac ultrasound), NG tubes (cardiac and vascular ultrasound, gastric ultrasound), G tubes (gastric ultrasound, urinary catheter (bladder ultrasound), nephrostomy tubes (urinary flow, kidney ultrasound), central venous catheters (vascular ultrasound, hemodynamics, flow, derivative flow, pressure, valvular closure, leakage or regurgitation), ports for therapeutic delivery and other suitable devices, or confirmation of tissue within a specific region or instrument (such as for valve clip devices, septal penetration devices, electrophysiology mapping instruments to confirm contact, or lung biopsy instruments for confirming the presence of solid tumors, nodes and/or non-aerated tissue).

As shown, the sensor portion 14 and an associated optical fiber 20 may be wrapped around the body 12. In order to secure the optical fiber 20 and the sensor portion 14 in place, as well as provide protection therefor, the ultrasound sensor wrapped around the body 12 may be covered by a protective coating, such as a conformal coating or the like, or may be covered by a protective sheath. The portion proximate the sensor portion 14 may consist of an acoustically sensitive material to further enhance the acoustic sensing, and increase bandwidth and detection range. Details on the tracking and imaging with the optical sensor can be found in a related U.S. patent application filed concurrently herewith, and assigned to the same applicant, titled “Medical Device Tracking with Shortest Distance Detection, as well as U.S. patent application Ser. No. 19/403,738, filed on Nov. 28, 2025, each of which is hereby incorporated by reference.

Alternatively, the body 12 may have a groove 24 formed in an outer surface thereof, with the ultrasound sensor 18 received within the groove 24, as best seen in FIG. 2B. As shown, as a non-limiting example, the groove 24 may be a helical groove. A helical-shaped groove minimizes tensile stresses in the optical fiber due to bending. As the optical fiber is routed along an outer surface of body 12 and secured using coatings, adhesives, or other bonding techniques, such configurations may enable exposure of the ultrasound sensor 18 to the surrounding environment for sensing, including acoustic sensing, imaging, and/or environmental parameter/physiologic measurement. Thus, the sensor may provide multimodality data or be calibrated to reflect temperature, interstitial pressure, or other biologic characteristics. Such an instrument may feed this data into a model, such as a finite element analysis model, to determine Pennes Bioheat equation effects upon tissue given a time temperature Arrhenius-like relationship, or a cumulative lethal tissue zone based upon known temperature effects over time. Alternatively, interstitial pressure data may be input towards an ultrasound-centered model with spatial coordinates for interstitial pressure, which may correlate somewhat with diffusion weighted imaging or attenuation diffusion coefficient (from a MRI). Such a map could also be used for registration purposes, since the spatial coordinates can be derived from the localization, and the point-based pressure measurements can be used to “map” or co-register spatial data, such that an MRI DWI/ADC map might be matched to 3-dimensional ultrasound or sensor data. It should be understood that multimodality sensing is not limited to this embodiment.

In the alternative of FIG. 2B, the groove 24 and the ultrasound sensor 18 may also be covered with a protective coating, such as a conformal coating or the like, or may be covered by a protective sheath 22, as shown in FIG. 2E. Such a sheath is preferably very thin and could, as a non-limiting example, be applied using heat shrinking, or an adhesive. The sheath may be made from, as non-limiting examples, polyamide, polyether ether ketone (PEEK) or the like. Non-limiting examples of such conformal coatings which provide additional protection for the sensor portion and the optical fiber include polyamide, parylene, silicone, acrylic, polyethylene terephthalate (PET), fluorinated ethylene propylene (FEP), polytetrafluoroethylene (PTFE) and the like.

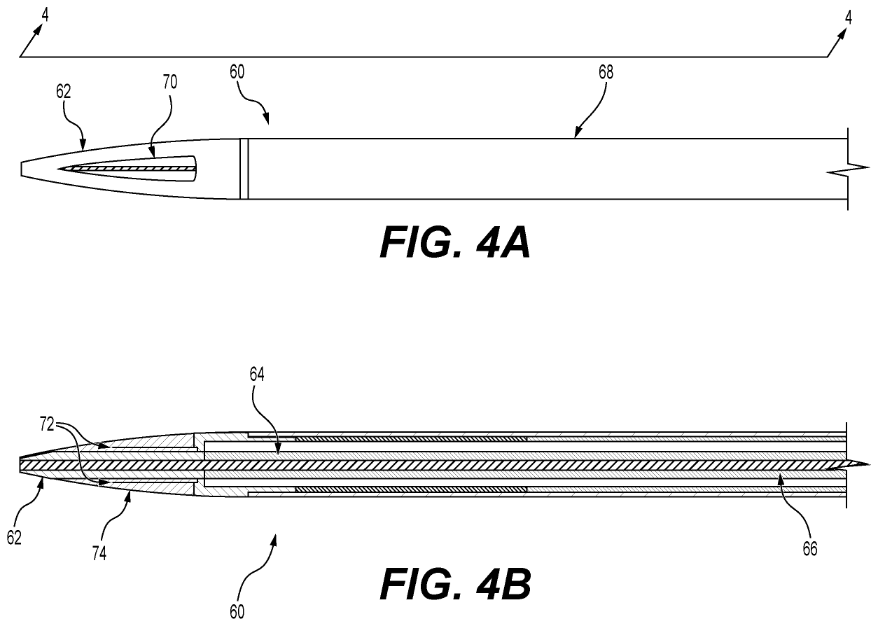

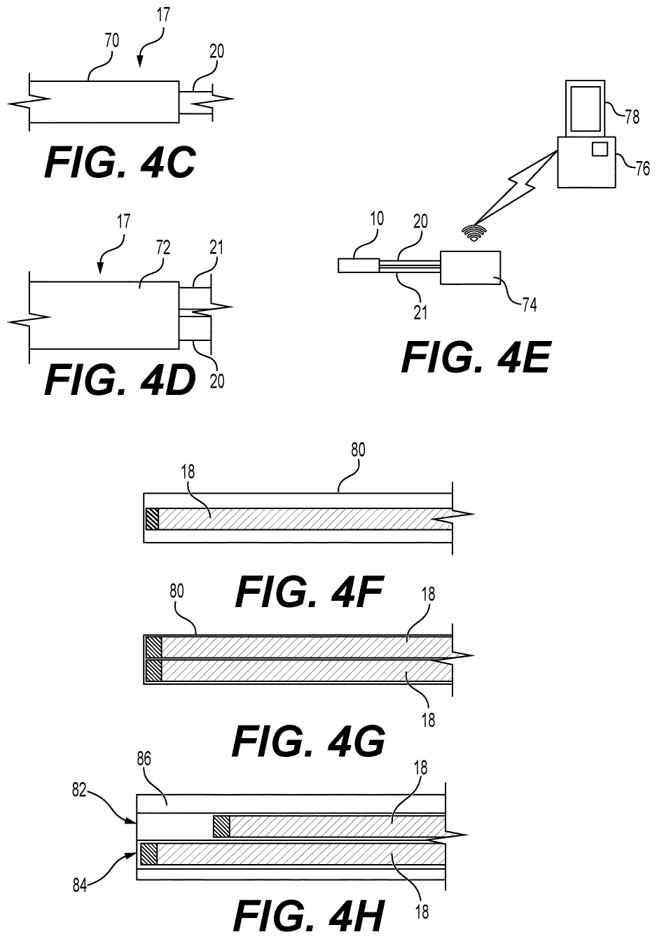

Further, as shown in FIG. 2B, two ultrasound sensors 18 (or more) may be mounted on the same body 12. As a non-limiting example, the two ultrasound sensors 18 may be wound around the body 12 diametrically opposite one another or, as a further alternative, two helical grooves 24 (or more) may be provided for the two (or more) ultrasound sensors 18. The two (or more) sensors 18 are at a known distance from one another. This distance may be lateral (as shown in FIG. 2B) or circumferential. As a further alternative, the two (or more) sensors 18 may be integrated with different materials for increased bandwidth and detection range within the same device (as shown in FIG. 4F).

As noted above, and as shown in FIGS. 1 and 2A-2D, the sensor portion 14 is positioned adjacent the distal end 16 of the elongated body 12. The distal end 16 may contain the working element of instruments for, as non-limiting examples, biopsy capture, ablation, angioplasty, intravascular or transcatheter procedures, and may or may not need a tapered tip, as noted earlier. In the non-limiting example of instrument 10 configured as an ablation instrument and incorporating the sensor 18, the distal end 16 of the body 12 is the working ablation element, thus although the sensor portion 14 is positioned in the vicinity of the ablation element, design considerations must address sensor performance in view of the thermal changes during an ablation procedure. The optical signal changes induced by the acoustic waves and temperature variation are on different time scales and can be separated with appropriate signal processing techniques, such as time-frequency analysis, filtering, or machine learning algorithms, allowing independent extraction of both dynamic acoustic responses and slower thermal effects for enhanced sensing precision. As discussed above, the sensor portion 14 and optical fiber 20 may be coated with a protective coating, such as conformal coatings, as noted above.

Further, it should be understood that the optical fiber 20 may be received within a lumen or other types of tubular structures, for example, with the lumen or the like being in contact with, or formed integrally with, the elongated body, or being received within the groove(s) 24. The optical fiber 20 may be slidable or moveable therein in order to prevent deformation thereof. As a non-limiting example, due to the heat generated by an ablation instrument 10, particularly in the vicinity of the ablation element, the body may expand. If the optical fiber 20 was rigidly constrained on the body, the thermal expansion and contraction of the body could cause deformation of the optical fiber 20. By allowing the optical fiber 20 to move within a lumen or other tubular structure, such deformation can be avoided. As non-limiting examples, such a lumen may be made integrally with the material forming the instrument body and/or from polyamide or PEEK lined with PTFE within the instrument body.

Although heated ablation is considered above, cryoablation tools have similar considerations; i.e., the differences in thermal expansion coefficients between the glass of the optical fiber, the material forming the body, and also the polymer coating or sheath can create stresses in the optical fiber, leading to microbending and signal loss. Thus, the lumen and/or the protective coating or sheath should be made from materials which remain flexible at low temperatures and, similar to the non-limiting example discussed above, the optical fiber should be free to slide and/or otherwise move within the lumen. In the example of cryoablation, if the optical fiber 20 is constrained, material contraction due to reduced temperatures can lead to fiber cracking. Additionally, in the non-limiting example of a cryoablation tool, the lumen and/or protective coating or sheath should be moisture-proof in order to ensure that no moisture can enter and subsequently freeze. A conformal coating of, as a non-limiting example, silicone may be applied as a moisture-proof coating. Thermal cracking might be avoided by precise placement of the sensor proximal to the therapeutic zone volume on a probe in order to better define edge effects of the ablation, where the temperatures are less extreme and where the information, such as treatment margin, is valued (i.e., the margin edge between ablated and non-ablated volumes). Such information may feed into ablation confirmation models or software, which may correlate with outcomes, as well as localize and define tissue or tumor volumes at risk for undertreatment.

Ablation procedures may benefit from the increased location accuracy provided by the on-tool optical-based ultrasound sensors described above to ensure that the ablation instruments are appropriately located within an ablation target. In addition to the increased location accuracy, real-time or near real-time tracking or location monitoring may be beneficial to ensure that the ablation instrument 10 is not displaced from the target location during a procedure. Further, optical-based ultrasound transducers may be provided at the distal ends of optical fibers that are non-conductive of RF and microwave energy. Thus, the radio frequency (RF) and microwave signals generated for ablation procedures do not interfere with the optical signals used in the optical based ultrasound transducers. Some location technologies that include traditional conductive wires may suffer from interference from the ablation energy. Traditional ultrasound systems that transmit and receive acoustic signals and photoacoustic or all-optical ultrasound systems also have limitations from a performance standpoint and the extent of image generation at the site of the ablation procedure. For example, gas bubbles may form during an ablation procedure, confounding the ability to image the ablation area. It should be understood that the optical sensor 18 may be used in conjunction with such ultrasound transmitters. Optical sensor 18 is able to receive ultrasound transmissions from an externally positioned probe, internally positioned probe (transesophageal echocardiogram (TEE), intracardiac echocardiography (ICE), intravascular ultrasound (IVUS), intraoperative ultrasound probe, endoscopic ultrasound (EUS), etc.) or an ultrasound patch. The ultrasound image will show the tissue changes in view of the ablation procedure. The optical sensor used for generating an ultrasound image may or may not be within the ablation thermal zone during the procedure and, thus, appropriate acoustically sensitive materials selected for the sensor must take into account the expected temperature changes. In addition to material considerations, the processor sends control signals to adjust the laser tuning parameters so that the optical sensor is able to continue sensing real time imaging despite the thermal changes and enable OnPoint imaging during the ablation procedure. The presence of gas bubbles that form during the ablation procedure, sometimes referred to as an “ablation cloud,” that limit real time conventional ultrasound imaging of the ablation area do not limit real time ultrasound imaging with the optical sensor. Real time ultrasound imaging with the optical sensor during the ablation procedure is minimally affected by the ablation cloud and with an understanding of any laser tuning requirements, making any necessary adjustments throughout the procedure, and the location of the optical sensor distal, proximal or within the ablation zone. It is noted that OnPoint imaging and any ultrasound-based imaging is affected by the ablation cloud, however, the present systems are far less susceptible to such issues. The co-pending and co-assigned U.S. Patent Application titled “Fiber-Optical Sensor System for Facilitating Ablation Procedures,” filed concurrently herewith, as well as U.S. Provisional Application No. 63/779,812, filed on Mar. 28, 2025, each further describe the implementation of acousto-optical sensors with ablation tools.

Additionally, the on-tool optical based ultrasound transducers may be used to measure temperature, e.g., as described in U.S. Patent Publication No. US 2024/0358256 A1, which is hereby incorporated by reference, or to measure other physical parameters. Such temperature measurements may be used to determine the extent of the ablation effect; e.g., based on tissue temperature. Such temperature measurements may be used to track and identify the boundaries of tissue isotherms created during an ablation procedure. In some embodiments, optical sensors may be provided on the ablation instrument and/or probes placed at the desired boundaries or targets of interest, enabling the clinician to receive real time ablation zone information, including image, temperature, impedance, pressure, and other tissue ablation parameters. Temperature measurement via optical sensors may have advantages over more traditional temperature transducers for several reasons. First, by using optical sensors for temperature measurement, it may be unnecessary to provide additional temperature sensors and wiring, which may lead to a bulkier ablation tool. Next, as discussed above, optical-based sensors are robust against interference from the RF and microwave energy generated during an ablation procedure.

It should be understood that any ablation procedure may benefit from the on-tool optical-based ultrasound transducers described herein. One example of such includes thyroid ablation. Thyroid ablation may particularly benefit from the tools and techniques described herein due to the unique challenges associated with such ablations. First, because of the non-circular shapes of thyroid nodule ablation targets, a “moving shot technique” is often used. The moving shot technique is used to move the ablation tool during the ablation procedure to create multiple ablation units, thereby permitting the interventionalist to shape the ablated area. Enhanced location accuracy and tracking permitted by on-tool optical-based ultrasound transducers can facilitate and improve this technique by increasing the ability to visualize and track the ablation tool and visualize the anatomy from the perspective of the sensor (OnPoint imaging), as well as providing real time ablation monitoring for the ablation procedure. Further, the thyroid is a relatively small portion of tissue with many adjacent structures that an interventionalist tries not to damage; e.g., the vagus nerve, the esophagus, the common carotid artery, the inferior jugular vein, the anterior jugular vein, the trachea, and the so-called “danger triangle” including the recurrent laryngeal nerve. Careful and accurate location of the ablation tool, and imaging of the surrounding anatomy from the perspective of the sensor (OnPoint imaging), facilitated by an on-tool optical-based ultrasound sensor, may aid the interventionalist in avoiding these areas by monitoring the ablation progression in real time.

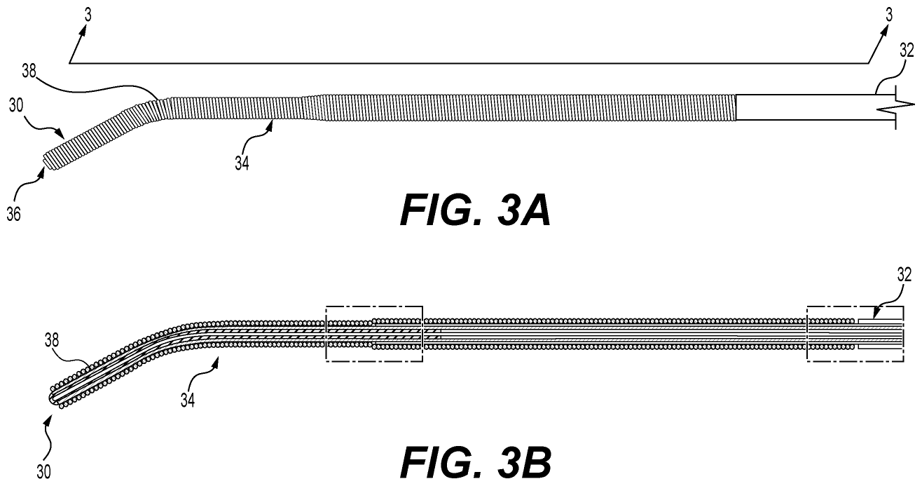

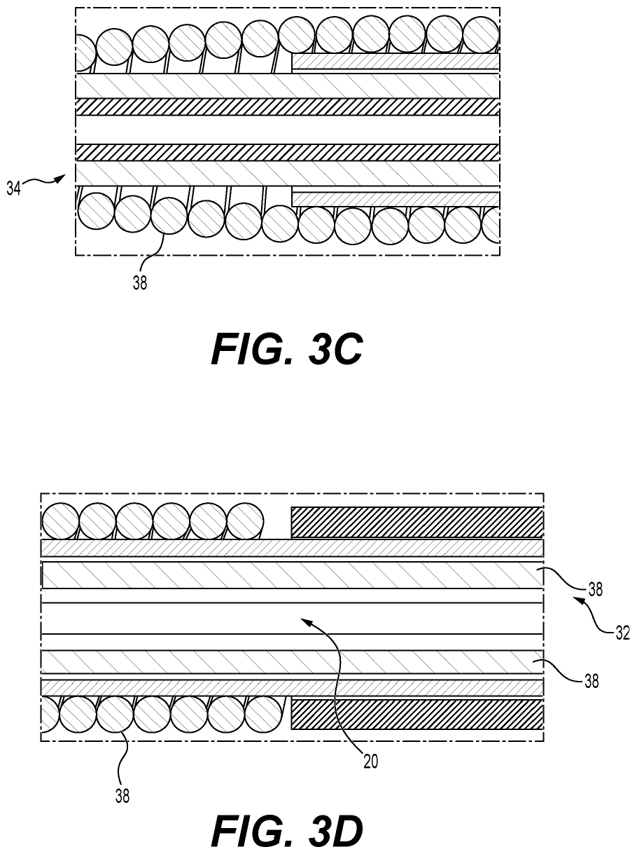

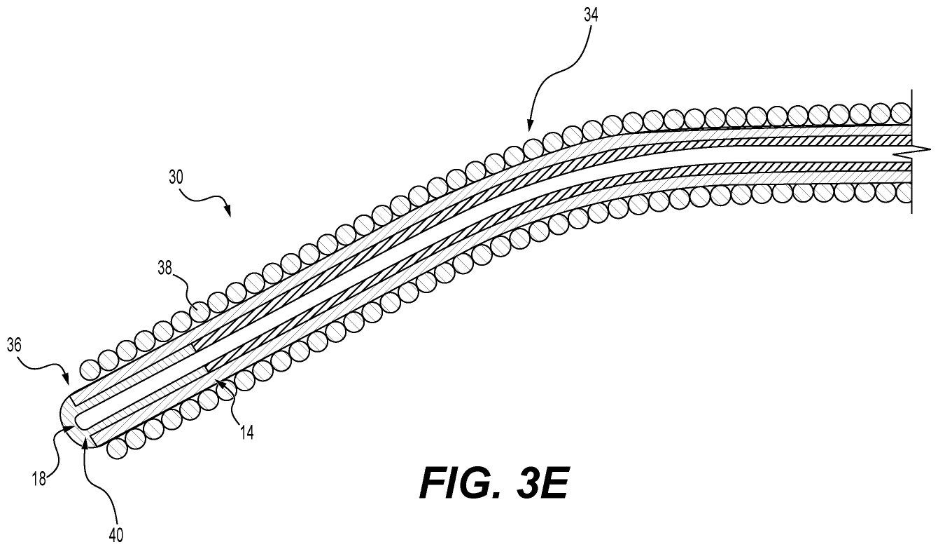

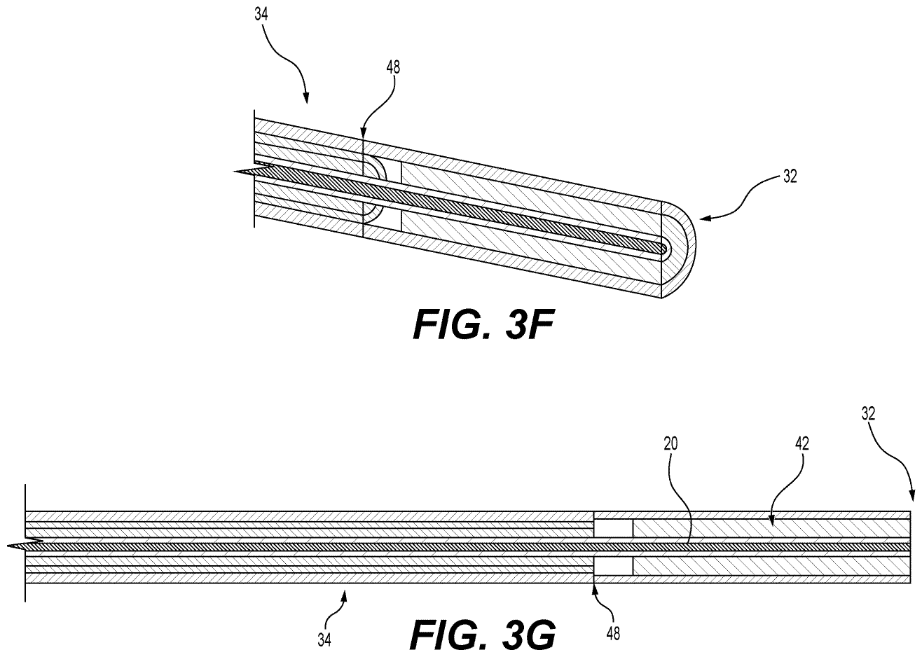

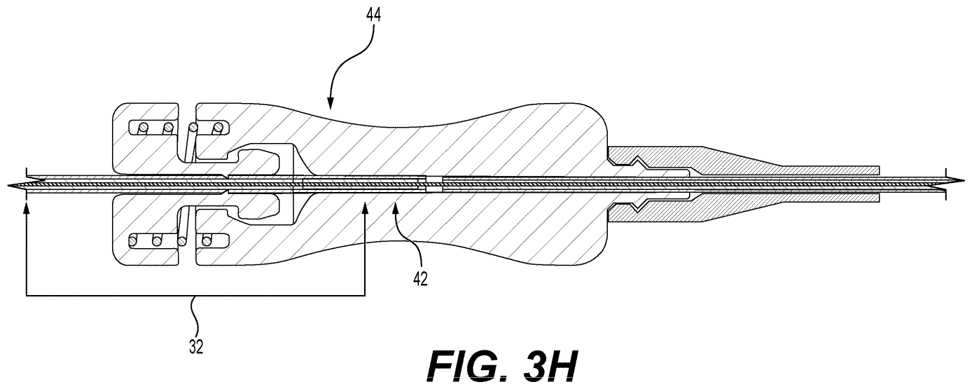

FIGS. 3A-3G, 4A and 4B illustrate another non-limiting example of medical devices with integrated ultrasound optical sensors, particularly incorporating ultrasound sensors into guidewires for catheter insertion or other instruments used for interventional procedures, such as endovascular device delivery systems, or intravascular therapeutic or diagnostic systems. Such procedures include Transcatheter Aortic Valve Replacement (TAVR) (aortic stenosis), transcatheter mitral value replacement (TMVR), embolic protection filters during operations, treatments for vascular disease (coronary or pulmonary artery disease), such as stenting, balloon angioplasty and atherectomy, treatment for endovascular embolization (aneurysms), and transcatheter edge to edge repair (TEER) (mitral valve clips (mitral regurgitation)). In general, guidewires are utilized for access, navigation, crossing of a stenosis or chronic total occlusion (CTO), and to deliver treatment or obtain diagnostic information. Common guidewire sizes may be 0.014, 0.018, and 0.035 inches in diameter. Smaller diameters allow for increased flexibility and are ideal for crossing high-grade stenoses. Larger diameters provide more rail support and tend to straighten tortuous vessels to allow for optimal delivery of devices for treatment or diagnosis.

FIGS. 3A-3G illustrate a guidewire 30 which, as a non-limiting example, may be used in transcatheter aortic valve replacement (TAVR) procedures; i.e., procedures to replace a diseased aortic valve with a man-made valve. Such guidewires typically include a relatively thick proximal end, which may be variable in length, and which remains outside the patient. The proximal end body 32 is typically relatively stiff and resistant to kinking, commonly about 0.035 ″ (0.89 mm) in diameter for TAVR procedures. 304V or 316L stainless steel is a common stiffener used for TAVR. Nitinol may also be used. This superelastic nickel-titanium alloy is more commonly used for neurovascular applications and requires additional radiopaque markers (typically platinum or gold). Hydrophilic (polyvinylpyrrolidone (PVP) or poly(ethylene oxide) (PEO)) or polytetrafluoroethylene (PTFE) coatings are commonly used for lubricity. The distal body 34 tapers and transitions from a stiff body to a flexible tip. The distal tip 36 is flexible, has a relatively small diameter, and is shaped dependent on its particular function. The thin tip is often wound with a coiled stainless steel wire 38 to increase diameter without adding stiffness. For a working guidewire, it should be understood that wires with different diameters (e.g., 0.014″, 0.018″, 0.035″) may be required, and that additional design parameters must be considered, such as the fiber diameters which will allow surrounding a fiber with a jacket, and the additional diameter and/or proximity/distance required for PZ elements and/or other transducer elements integrated into the fiber.

A fiber-end sensor may be as small as approximately 50 microns. The optical fiber on which the sensor resides can have an outer diameter as small as ~80 microns (~0.0031″), however, a typical size of 125 microns is often used, including the glass fiber and polyimide coating. At this scale, a unique integration within a 0.014″ guidewire form factor is most feasible, with headroom for a surrounding jacket and mechanical reinforcement. Smaller diameter wire formats (such as for neuro applications, for example) are also technically possible, subject to final jacket and handling requirements. Such a small sensor integrated into a guidewire (via mandril or peripheral integration) enables imaging, data, and tissue interrogation with less risk of inducing thrombus, extravasation, or rupture of the lumen or cavity, or damage to nearby anatomy. Larger sensors and imaging devices are not used due to the risk of operating such larger sensorized devices in vulnerable clinical anatomy and scenarios.

For a hollow guidewire, such as that shown in FIGS. 3A-3H, an internal ultrasound sensor, such as those discussed above, may be incorporated in order to localize and guide the guidewire 30. FIG. 3D best shows optical fiber 20 of the sensor 18. In order to integrate the ultrasound sensor 18, the inner diameter of the guidewire 30 needs to be minimized in order to maximum stiffness of the guidewire, but still allow sufficient clearance for the fiber. There should also be a relatively small amount of space between the ultrasound sensor 18 and the inner wall 38 of the guidewire 30 to allow for bending during navigation without putting damaging stress or strain on the optical fiber 20. The sensor portion may be located at the very tip of the guidewire 30 or may be positioned approximate the distal end within the guidewire lumen or potted within windows, which may be laser-cut or the like. FIG. 3E shows the sensor portion 14 secured using a potting compound 40. As in the previous embodiments, two or more such ultrasound sensors 18 may be incorporated into the guidewire 30. As shown in FIGS. 3F and 3G, the distal body 34 and the proximal body 32 may be joined, as a non-limiting example, at a laser welded seam. For purposes of clarity, coiled wire 38 is not shown in FIGS. 3F and 3G. Further, optical fiber 20 is shown mounted in a ferrule 42 within the proximal body 32 in FIG. 3G.

In use, once the guidewire 30 is advanced to the desired position within the patient, the optical fiber 20 must be removed at the proximal end 32 so that the catheter can be introduced. In one embodiment, as shown in FIG. 3H, the proximal end 32 of the guidewire 30 may be a fiber ferrule 42 without the typical connecter subassembly. A mating connecter subassembly 44 that attaches to the jacketed fiber/system connector can be slid over proximal end 32 of the guidewire 30. The mating connector 44 may be similar to push connectors for semi-rigid tubes, which are pushed into firm connection and connection is released by press of a coupled button, allowing the connector to be removed. In another embodiment, a connector may be integrated at the proximal end 32 of the guidewire 30, which has a permanent breakaway or detachable section. During manufacturing, the optical fiber 20 can be weakened at a specific point within the connector using a micro-etching or laser ablation technique, as examples. The connector can be designed so that applying a controlled bending or twisting force at this point would cause the fiber to break cleanly. As a further alternative, an integrated cutting mechanism may be used to create a clean break in the optical fiber. Once the guidewire 30 is in its desired position within the patient and imaging using the ultrasound sensor is complete, the operator can detach this proximal part, leaving a smooth guidewire body for catheter advancement. Depending on the nature of the delivery catheter (such as an over the wire or rapid exchange configuration), the optical fiber may be reattached. This will allow for OnPoint imaging and/or environmental or physiological sensing during the procedure. The inherent flexibility of this sensorized approach near or at the tip distal most of a guidewire, and incorporated into various shapes, is also an enabling feature.

Robotic surgical systems or other interventional procedures may use fiducials to assist in the accuracy and reproducibility of surgical procedures by providing real time monitoring of the surgeon's execution. Similar to a guidewire, a sensorized fiducial, commonly used with robotic surgical systems, may consist of an elongated optical fiber with an acousto-optical sensor at its fiber end (or other desired location) along the distal portion. Acousto-optical sensor fiducials may be delivered through a catheter or other tool and be left in place during the procedure. The optical cable may be operably connected to a hub containing the OSoC or a conformal coating may be applied to both protect the fiber and provide a robust elongated fiber that may be delivered through a catheter or introducer to the desired location. The catheter or introducer may be removed, leaving the fiducial in place during the procedure. The fiducial may need a more robust form factor and take on the form factor similar to a guidewire, catheter or thin stylet or probe. The proximal end of the fiducial's optical cable will need to be connected to a laser source, a photodetector (PD) and system processor in a similar fashion as the other sensorized instruments described herein.

In other embodiments, a temporary indwelling fiducial can colocalize a device, such that other imaging types (e.g., fluoroscopy, CT, CBCT, ultrasound or MRI) may localize the device and allow for co-registration and fusion or co-display. The 5th and 6th degrees of freedom may be obtained by multiple sensors, or the addition of another system, such as a gyroscope, MEMS device, or electromagnetic tracking.

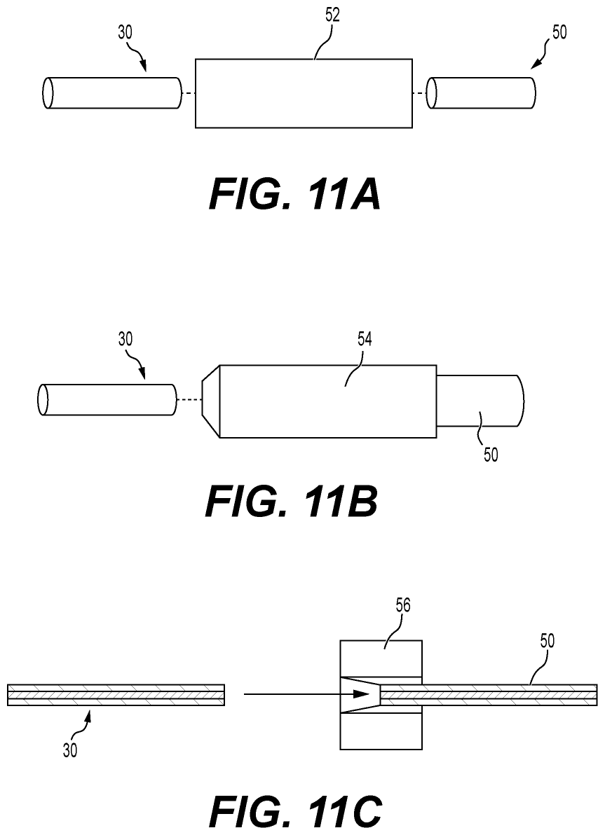

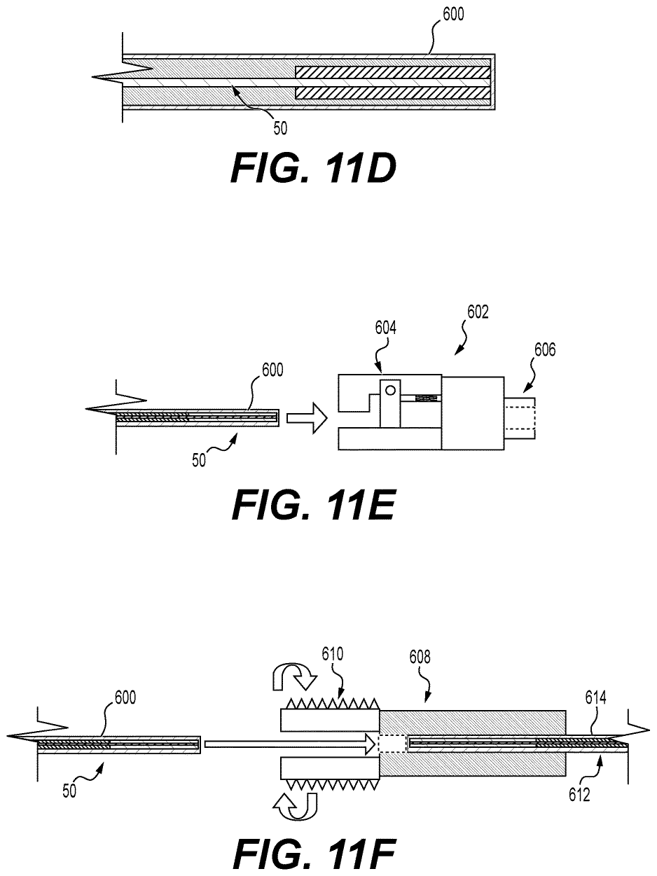

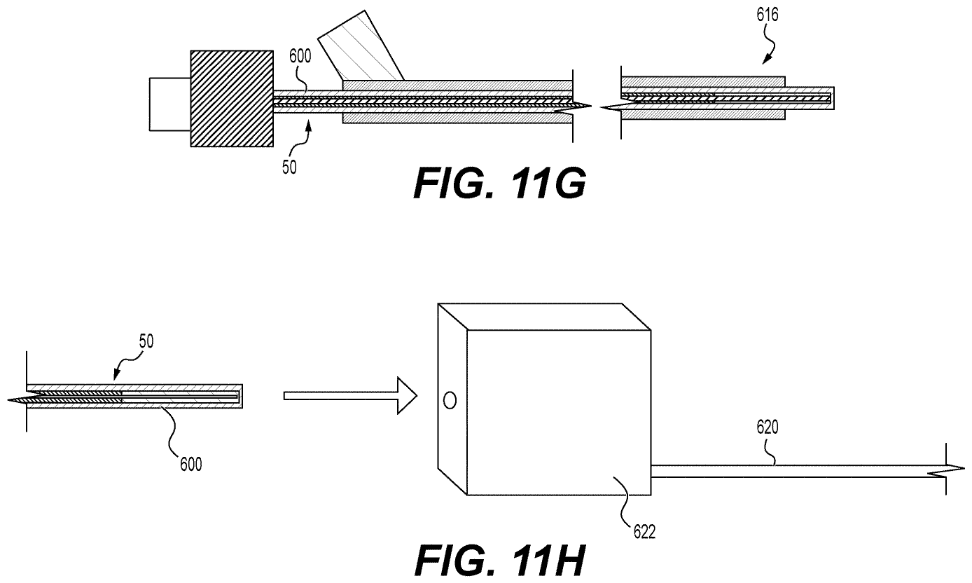

As a non-limiting example of such a connector, FIG. 11A illustrates a bare fiber 50 with a bare fiber terminator 52. The sensor is used with a bare fiber terminator 52 to connect to the console. The bare fiber terminator 52 can then be disconnected and the stent (or other instruments) can be inserted. The bare fiber 50 is then reinserted into the bare fiber terminator 52 and the sensor is reconnected to the console. In the alternative embodiment of FIG. 11B, a bare fiber 50 is used with a screw-type terminator 54. The sensor is used with a screw-type terminator 54 to connect to the console. The screw-type terminator 54 may then be disconnected for insertion of the stent (or other instruments). The bare fiber 50 is then reinserted into the screw-type terminator 54 and the sensor is reconnected to the console. FIG. 11C illustrates another non-limiting example, in which a spliced standard fiber connector 56 is used with the sensor to connect the sensor to the console. The fiber may be cut for insertion of the stent (or other instruments). Other connection mechanisms are well described in other delivery systems and may be adapted for use in connection, disconnection and reconnection on the back end of a working wire. This includes electrolytic dissolution of a connection via electrical current or rotational unscrewing or bending of an enabling required back ended element.

FIGS. 11E-11H illustrate further examples of fiber connectors which may be utilized. In each of these non-limiting examples, as shown in FIG. 11D, the optical fiber 50 is partially contained within a fiber ferrule 600, which may be made of Nitinol® or the like. In FIG. 11E, the optical fiber 50 is shown connecting to a bare fiber terminator 602, which may form a tight connection with optical fiber 50 via a spring-loaded clamp 604. At the other end of the bare fiber terminator 602 is a standard fiber connector ferrule, which may be used for mating with a standard fiber connector (FC, SC, ST, etc.) from the back-end system, along with any necessary adaptor.

FIG. 11F shows another non-limiting example, in which a screw-type fiber connector 608 is used, where rotating tightener 610 receives the optical fiber 50 and is rotated to clamp the optical fiber 50 once optical fiber 50 is in contact with the additional optical fiber 612 (shown contained in its own ferrule 614). FIG. 11G illustrates the optical fiber 50 pre-loaded into a non-limiting example of a medical device 616 (such as any of the medical devices discussed herein), with the proximal end of the optical fiber 50 connected to a standard fiber connector 618 (FC, FSC, ST, etc.).

FIG. 11H illustrates optical fiber 50 being inserted into an automated fiber alignment machine 622 for connection to additional fiber 620. Such machines use internal sensors and motors (or other types of actuators) to automatically align the two facing ends of the optical fibers for connection. As a non-limiting example, alignment may rely on the reflection intensity feedback loop control of the motors reaching maximum reflection intensity. As a non-limiting example, such an automatic alignment machine 622 could be incorporated into a tool used with a robotic system. For example, the Hugo™ RAS system, manufactured by Medtronic®, offers nine compatible instruments, including monopolar and bipolar options, needle drivers, and graspers. The sensors described herein could be integrated into one such instrument, forming a sensorized instrument, or any other sensorized instrument which could be handled robotically, and further include the automatic alignment machine 622 for making automatic optical fiber connections.

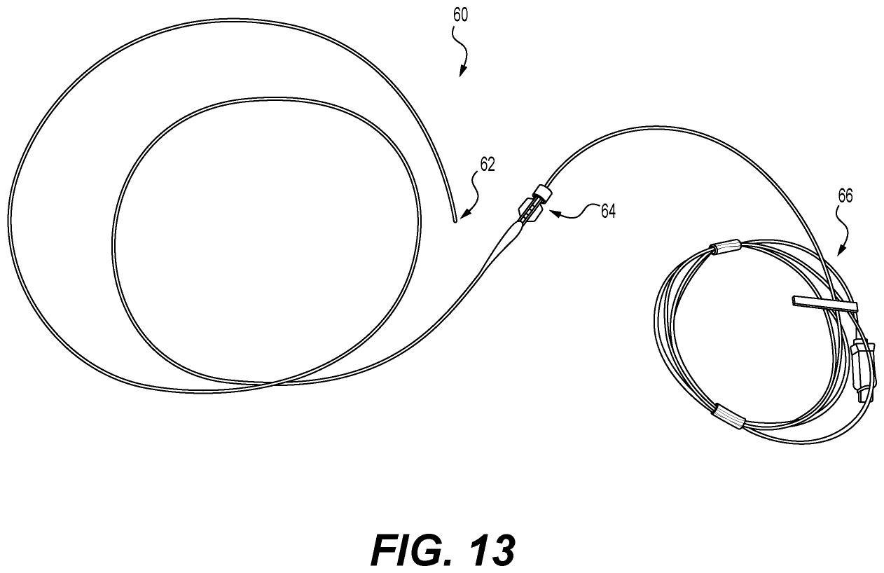

FIG. 13 shows a catheter 60 with a sensor distal end 62 and transition region 64 for selectively coupling the catheter 60 to the laser source via the fiber cable 66. In the non-limiting example of FIG. 13, a 1.8 Fr sensorized catheter 60 incorporating a fiber-end sensor (FES) and a transition region 64 is shown, allowing for the coupling and decoupling of the fiber-end sensor 62 to the laser source, thereby permitting coaxial introduction of an instrument over the guidewire.

Since OnPoint imaging (i.e., creating ultrasound images from the perspective of the optical sensor location in situ, such as, but not limited to, B-mode, color doppler, shear wave, PW doppler, etc.) relies on the sensor being able to receive the beamformed acoustic signals. The acoustic ring may allow for greater signal reception, regardless of the catheter orientation. Alternatively, multiple sensors may be oriented on the tool or in an array format so that, regardless of its orientation, sufficient signal is received by the sensor for tracking and OnPoint imaging for various imaging modes and environmental or physiologic sensing. However, this may also be accomplished in part or in whole by rotation of a sensor in a known rotation (relative to the axis). The beamformed signals may be generated from a variety of probes located external or internal to the body, including a transesophageal echocardiography (TEE) probe or an intraoperative ultrasound transducer used in minimally invasive or other surgical procedures as the transmit source for On-Point imaging for image quality better than an ultrasound (US) excitation from the probe outside the body. Although acoustic signals transmitted from a probe positioned on the exterior surface of the body are also suitable. Integration of the ultrasound source may further be combined with a wire or catheter or sheath to provide ultrasound from a known location and spacing to the optical sensor. Signal deconvolution approaches may be used to reduce internal ultrasound propagation and/or resonance inside the acoustic ring, improving the signal homogeneity for OnPoint imaging.

The guidewire may be manufactured with a variable outer diameter with a small, relatively constant inner diameter. Alternatively, the guidewire can be manufactured with a constant wall thickness but with a variable inner diameter. This latter alternative would be easier to manufacture but would result in a guidewire with less stiffness. As shown in FIGS. 3A-3E, concentric tubes may be used in the manufacture, allowing for a stepped variable thickness within the tapered section. These tubes may be laser welded in place, for example, and wrapped with, for example, 0.003″ diameter stainless steel wire from the tip to the distal end of the proximal body.

As a further alternative, the guidewire 30 may be solid and an external ultrasound sensor may be integrated therewith. As a non-limiting example, the ultrasound sensor may be wound about the guidewire 30, similar to the previous embodiments. A helical winding could be used for the ultrasound sensor, as a non-limiting example. In order to secure the ultrasound sensor to the guidewire 30, a relatively flexible, ultraviolet (UV) curable coating, for example, could be applied, preferably with a constant thickness which is slightly larger than the diameter of the optical fiber. Similar to the previous embodiments, a groove, such as a helical groove, could be formed in the outer surface of the guidewire for receiving the ultrasound sensor. In this alternative, the UV curable coating may also be used, but with only the groove filled with the coating. As a further alternative, a flexible outer sheath with the ultrasound sensor embedded therein may be applied to the guidewire. A final hydrophilic coating may be applied in order to provide lubricity. However, it is noted that the diameter of the guidewire may have to be reduced in order to accommodate the added thickness of the ultrasound sensor and the coating. Manufacturing will also have to take care to produce a device with a smooth surface with sufficient cylindricity throughout its length.

FIGS. 4A and 4B illustrate a delivery catheter 60 with a guidewire. The catheter 60 may include a soft distal nosecone 62, such as, as a non-limiting example, a silicone nosecone with a 30-40 Shore A durometer hardness. The catheter 60 has a flexible inner core 64 with a central lumen 66 for the guidewire. The central lumen 66 may be made from PTFE, as a non-limiting example, or similar materials. The flexible outer sheath 68 may be made from, as a non-limiting example, a stainless-steel braid or coil coated with polyurethane (PU), polyethylene (PE), silicone, nylon, Pebax® (a block copolymer variation of polyether block amide (PEBA)), or other polymer or metallic or biogel coating material. The catheter 60 may include a handle with a sheath retracting mechanism or other configurations for operating the distal end of the delivery catheter, along luer connections, and may be used with, dependent on the particular application, radiopaque markers.

An additional off-center lumen may be provided within the core 64 for receiving the ultrasound sensor. As with the previous embodiments, two or more ultrasound sensors (and two or more corresponding lumens) may also be used. The nosecone 62, as shown, has windows 70 integrated therein, and the sensor portions 72 are positioned in the windows 70. The windows 70 may then be filled with a potting compound 74. Depending upon the nose cone material and thickness, and the intensity of the ultrasound transmission, a window 70 may not be needed, as the signal-to-noise ratio (SNR) or the signal strength of the sensed acoustic signal will be sufficient. As an alternative to a pair of sensors, a single sensor with an acoustic ring may be incorporated into the nosecone. Since OnPoint imaging (i.e., creating ultrasound images, such as, but not limited to, B-mode, color doppler, shear wave, PW doppler, etc.) relies on the sensor being able to receive the beamformed acoustic signals, the acoustic ring may allow for greater signal reception, regardless of the catheter orientation. The beamformed signals may be generated from a variety of probes and, as noted above, multiple sensors may be oriented on the catheter or in an array format such that sufficient signal is received by the sensors for tracking and OnPoint imaging for various imaging modes and environment or physiologic sensing.

Compared with the guidewire procedure discussed above, use of the catheter 60 of FIGS. 4A and 4B does not require detaching the optical fiber at any point in the procedure. The catheter 60 is also easier to manufacture and assemble, and allows for integration of one or more sensors for directionality, triangulation, redundancy, and the like. Additionally, there is an exact correlation between the ultrasound sensor and the deployable device position.

FIGS. 4C and 4D illustrate the proximal end 17 of the sensor-carrying instrument 10, which may include only an optical connector 70 for the ultrasound sensor (FIG. 4C) or may include dual optical and electrical connectors 71 (FIG. 4D), or may include optical, electrical and fluid connectors. In FIG. 4C, only optical fiber 20 is connected, but FIG. 4D shows both optical fiber 20 and an electrical wire or pad 21 for providing power and/or delivering electrical sensor signals. These connectors are used to connect or tether the instrument to one or more control units (e.g., ultrasound system, other medical imaging system (MRI, OCT, CT, fluoro) ablation (cryo or heat)). It should be understood that the electrical wire or pad 21 may communicate with any suitable associated electronics, such as those provided for receiving data from the ultrasound probe, the transponder, and/or an inertial momentum unit (IMU). It should be understood that the associated electronics encompasses any suitable type of computer processor, controller or the like for utilizing and processing the data for generating an ultrasound image and tracking the medical device in the patient. An associated display may also be provided for visualizing the body region for the medical procedure and the location of the medical device.

For the hybrid connector 72, the optical fiber 20 can carry the optical signal and the electrical cable 21 can carry the control signal, sensor data, or additional electrical sensor signals. Alternatively, as shown in FIG. 4E, wireless connections may also be implemented by using a wireless catheter proximal hub 74, which includes a light source, such as an optical system on-chip (OSoC), optical components/modules, photodetectors, optoelectronics, and a wireless communication module, for example. An OSoC may be used for the optical source and, in order to maintain its tuning, be isolated from other components in the hub 74. It should be understood that the backend system 76 in wireless communication with hub 74 is shown for exemplary purposes only and, as discussed above, may be any suitable type of computer, server, computer processor, controller or the like. Similarly, the associated display 78 may be any suitable type of display.

As discussed above, and as illustrated in FIGS. 4F and 4G, the catheter may use one or two ultrasound sensors. FIG. 4F shows a single lumen catheter 80 with a single ultrasound sensor 18. FIG. 4G shows a single lumen catheter 80 with two ultrasound sensors 18. Alternatively, as shown in FIG. 4H, multiple lumens 82, 84 may be provided in the same catheter 86 for receiving multiple ultrasound sensors 18 in differing configurations and orientations. Although only two lumens 82, 84 are shown in FIG. 4H, it should be understood that three or more lumens may also be provided. In certain applications, as noted above, the sensor instrument 10 may remain within the patient for ongoing monitoring and/or imaging. In certain embodiments, the imaging or monitoring needed will be intermittent and thus connectors that are removably connected would be advantageous.

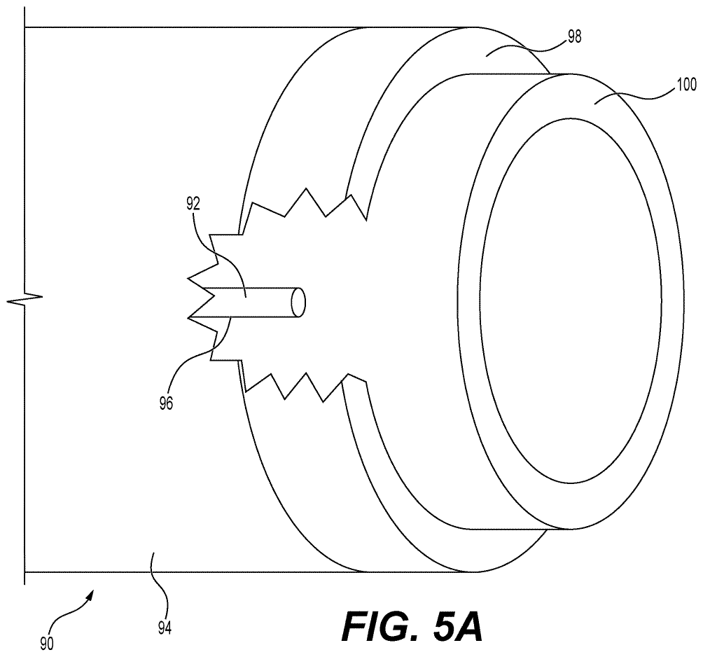

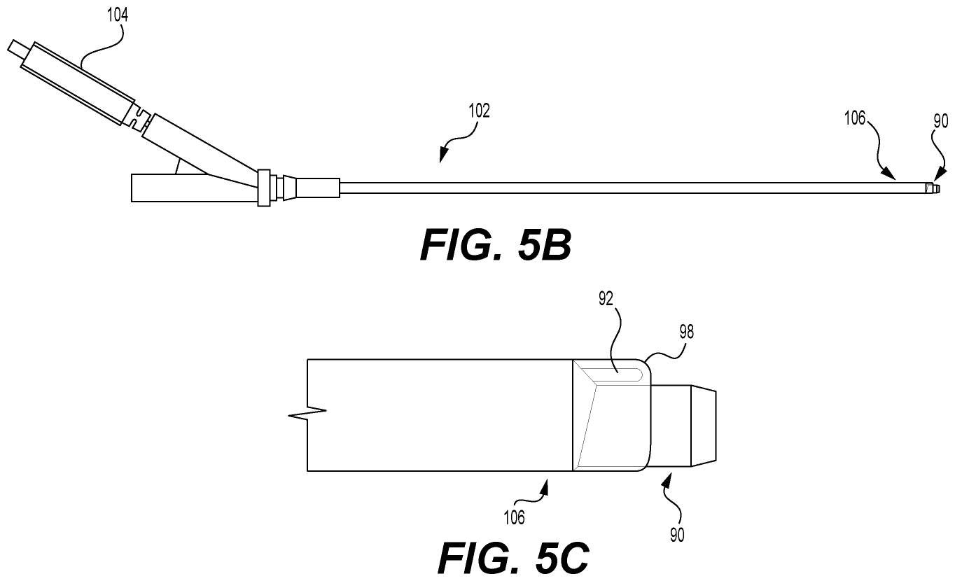

In the further embodiment of FIGS. 5A and 5B, a tubular-shaped medical device 90, such as a biopsy sheath, an introducer, a sheath, a trocar, a microcatheter, a catheter, an intravenous needle, instruments used in minimally invasive surgical procedures, instruments used with robotic assisted surgical systems or the like, has an integrated ultrasound optical sensor 92, such as the ultrasound sensors discussed above. As shown, the tubular structure 100 of the device 90, such as an introducer, as a non-limiting example, is covered with a cover layer 94 and the optical fiber 96 is embedded therein. The sensor portion 92 is embedded in an adjacent annular layer 98 formed from an acoustic matching material. It should be understood that the acoustic matching material may be formed from any suitable type of material for achieving appropriate acoustic impedance to maximize ultrasound transmission. The acoustic matching material is selected for acoustic impedance matching with the target environment to reduce acoustic reflections at the interface between the sensor portion and the target environment.

In FIGS. 5B and 5C, the medical device with an integrated ultrasound optical sensor 90 of FIG. 5A is implemented in a biopsy sheath 102, with the sensor portion 92 positioned adjacent the distal end 106 of the sheath and contained within an acoustic matching material 98, such as an acoustic matching potting compound or the like. It should be understood that the particular shape of the annular layer 98 and the particular shape of the device tip (i.e., the beveled tip in the non-limiting example of FIG. 5C) are shown for exemplary purposes only. As in the previous embodiments, the optical fiber 96 may be received within a lumen, with the lumen being embedded within the cover layer 94. The fiber optical connector 104 may be connected to the ultrasound system through an optical fiber cable. The ultrasound system contains the components for powering the optical sensor 92, converting the sensed ultrasound signals to data that can be then processed for imaging, visualization, tracking and environmental or physiologic sensing purposes. Alternatively, the optical connector 104 and fiber cable may be replaced with the OSoC described earlier. This will contain an OSoC, optoelectronic (O/E) components, a battery and wireless transmission components. As an alternative embodiment, it may contain an electrical cable for connecting the optical sensor and OSoC to the ultrasound system for data processing and providing tuning support for the OSoC.

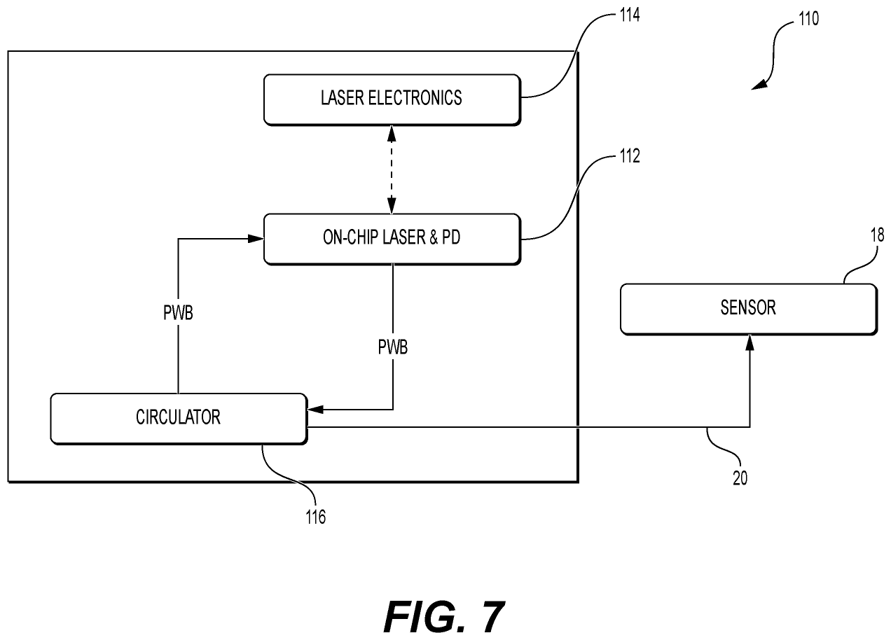

As discussed above, the medical devices with integrated ultrasound optical sensors may use one or more optical system-on-chips (OSoCs) as light sources for the ultrasound sensor(s). The OSoC may be integrated into, or mounted on or within, the device proximal hub. FIG. 7 illustrates an optical system 110 which may be integrated into, or mounted on or within, the device proximal hub of a medical device with an integrated ultrasound optical sensor, such as in any of the embodiments discussed above. The system 110 includes an on-chip laser and photodiode (PD) 112 in electrical communication with any necessary electronics 114 for the operation and control thereof, and in optical communication with an optical circulator 116. The optical circulator 116 is in optical communication with the one or more sensors 18 through optical fiber(s) 20. A typical size for a photonic integrated circuit (PIC) chip with an on-chip laser, on-chip PD, and an ultra-mini optical circulator is approximately 3.0 mm×20 mm. It should be understood that one or more on-chip lasers may be provide for respective coupling with one or more ultrasound sensors or, alternatively, a single on-chip laser may be provided for coupling to multiple ultrasound sensors. It should be further understood that any suitable type of optical coupling/communication may be used, such as, but not limited to, photonics-wire-bonding (PWB) and optical fiber(s).

It should be understood that any additional electronic, optical or electrooptical components may be used with the optical system illustrated in FIG. 7, either similarly mounted on or in the device proximal hub, or located separately from the device proximal hub. As a non-limiting example, a thermoelectric cool (TEC) may be used to cool the on-chip laser and any suitable type of electronic controller or control system may be in communication with the TEC and/or the on-chip laser for appropriate temperature control. Additionally, a controller may be provided for controlling the laser output power, as well as monitoring and controlling any noise in the laser output (relative intensity noise (RIN) and/or wavelength noise). Further, it should be understood that the on-chip laser may be powered by any suitable wired or wireless power source, such as, but not limited to, a rechargeable battery. Similarly, it should be understood that any suitable electronics for monitoring and controlling the PD also be coupled with the on-chip PD. As a non-limiting example, a controller may monitor the PD to detect any damage thereto, as well as monitoring and correcting DC saturation levels, AC noise levels or the like. The overall optical system may also be monitored for, as a non-limiting example, overall system optical loss (e.g., component insertion loss, coupling loss and the like).

In addition to the above, the ultrasound sensor(s) work in combination with a source of ultrasound signals. It should be understood that any suitable type of ultrasound source(s) may be used. A controller may be in communication with the ultrasound source(s) to control and monitor the transmission power, transmission sequences, etc. Further, with regard to the applications of the ultrasound sensor(s), such as instrument tracking, as a non-limiting example, a controller may be provided to receive the signals generated by the ultrasound sensor(s) and convert the signals into data representative of movement and location, which can then be displayed to the medical personnel.

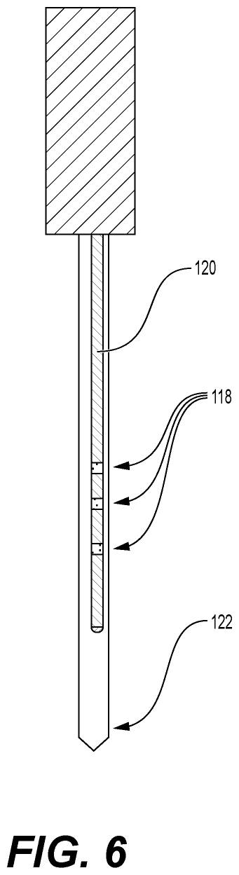

As discussed above, it should be understood that any suitable type of ultrasound sensors may be utilized. As a non-limiting example, as shown in FIG. 6, a number of fiber Bragg grating (FBG) sensors 118 may be etched, or otherwise formed, in an optical fiber 120, with one further tip sensor 122, such as those discussed above, at the tip end of the optical fiber. This allows for enhanced capability, such as providing thermal and/or pressure mapping at the same time that the tip end acousto-optic sensor 122 is performing in a manner such as that described above. This would allow for additional applications, such as, as non-limiting examples, use of a pressure sensor for regurgitation detection, FFR procedures, temperature sensing, or other environmental or physiological measurements. This may be further combined with membrane sensors, microfluidic Fabry-Pérot cavity sensors or the like. As a further alternative, the sensor(s) may be integrated with photoacoustic illumination for photoacoustic spectroscopy, which is used to evaluate heart disease/blood oxygenation, for example, or characterize neoplastic tissue from normal tissue in biopsy, ablation or other applications. As another alternative, the sensor(s) may be integrated with optical coherent tomography (+/−IVUS) for hybrid imaging. For this type of multi-modality imaging, the all-fiber-based OnPoint imaging may offer distinct advantages over conventional ultrasound (US) probes excited externally on the surface of the patient, or from interoperative, IVUS, ICE or TEE transducers. Acoustic lenses and/or mechanical spiral/steering structures may be used to scan the sensor through a region of interest to form an image, and multi-modality co-registration can combine functional information of the tissue, enhancing imaging and/or characterization. Multi-modality imaging with IVUS, ICE, TEE or other imaging probes also provides advantages by incorporating OnPoint imaging from the position of the sensor or sensors to supplement and expand upon the imaging from such transducers. This may also provide imaging in areas where such transducers have limitations, such as by allowing beamforming through acoustic challenging regions (e.g., ribs) with the OnPoint acoustic geometry.

With regard to OnPoint imaging, as a non-limiting example, OnPoint imaging could be used to identify calcification on valves, particularly by detecting a strong reflection of a calcified valve, thus making calcifications easy to differentiate from valve/tendons. Such an approach could also be useful in localizing or identifying vulnerable components of plaque with lipid content changes in sensed properties. OnPoint can also be used to identify the valve location for, as a non-limiting example, rough placement of TAVR, TMVR or TEE to reduce fluoroscopy time. The use of this ultrasound-based system reduces radiation exposure for physicians.

As noted above, OnPoint doppler may be used for regurgitation detection, as a non-limiting example. Regurgitation after valve placement is a routine check for TAVR, TMVR or TEE and using OnPoint doppler provides the ability to pick up lower velocity regurgitations. The imaging frame rate could be increased for more at-valve visualization by skipping the tracking and/or B-mode frame. The frame rate requirement in heart US imaging is higher due to heart motion, and skipping the B-mode and tracking frame allows for a faster refresh rate. Additionally, acquiring only the signal from the sensor time of arrival to the region of interest being visualized can significantly increase the frame rate. As another example, shear wave imaging also requires a high frame rate. The shear wave created by the patient's beating heart could be used for imaging. The measurement of myocardial stiffness can provide insights into cardiac pathophysiology. As a further alternative, a directional fiber sensor could also be used for shear wave enhancement. It should be understood that the various sensors discussed above may be used in combination with, or integrated with, any suitable optical or acousto-optic components, such as acoustic lenses and the like. The sensor could be used to inform an ultrasound DWI/ADC-like map, as previously described.

As discussed above, a chip-based laser (OSoC) or a fiber-based laser may be coupled to the device proximal hub, or embedded therein, providing an on-device light source for the ultrasound sensor, which is in communication with the chip-based laser or fiber-based laser. The chip-based laser is particularly well-suited for sensors requiring multiple light sources simultaneously. In other embodiments, the instrument optical sensor may be tethered via optical cables to a laser located at a system controller housing a laser, optical/electronic components and data processing. In the non-limiting example of FIGS. 1 and 2A-2E, the elongated body of an interventional instrument is shown with the sensor portion positioned adjacent a tapered distal end. While the distal tip is shown tapered, the actual geometry will depend upon the instrument. The interventional instrument may be a working guidewire over which are placed instruments for stent graft deployment and/or sizing, balloon deployment, recanalization or crossing device, thrombectomy or other instruments used for conducting interventional procedures, such as a transcatheter procedure instrument (such as transcatheter aortic valve replacement (TAVR), transcatheter mitral valve replacement (TMVR), or transcatheter edge-to-edge repair (TEER)) or other minimally invasive/interventional procedures.

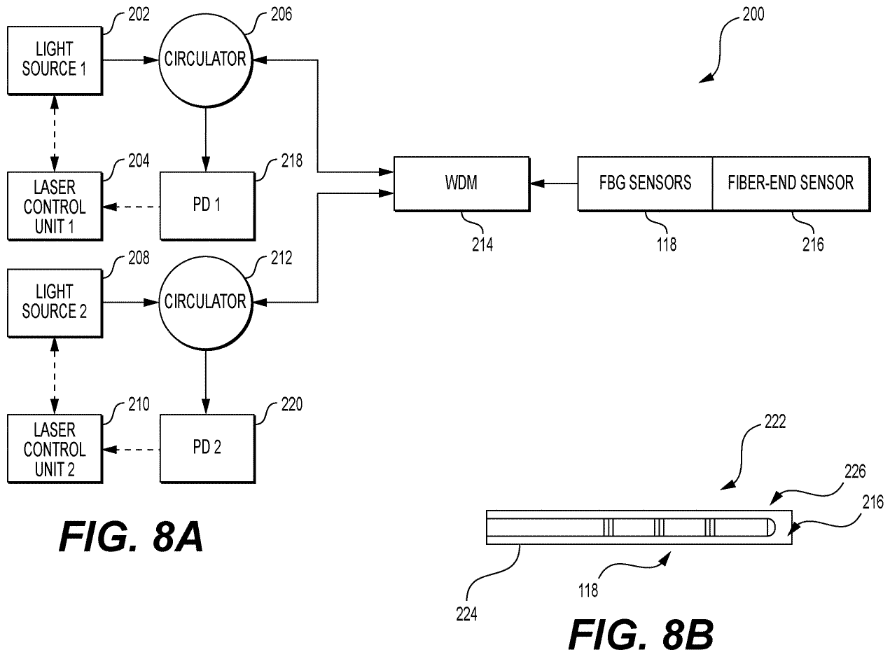

FIG. 8A diagrammatically illustrates a system 200 for use with a hybrid optical fiber sensor. A first light source 202, such as a laser, is under the control of a first laser control unit 204. The first light source 202 generates a laser beam at a first frequency, which is fed to optical circulator 206. Similarly, a second light source 208, such as a laser, is under the control of a second laser control unit 210. The second light source 208 generates a laser beam at a second frequency, which is fed to an optical circulator 212. Although only two lasers operating at two different wavelengths are shown in FIG. 8A, it should be understood that this is for purposes of clarity and simplicity only, and that a relatively large number of beams at different wavelengths may all be generated, passing through corresponding circulators for multiplexing by a wavelength division multiplexer (WDM) 214. In FIG. 8A, the sensor system is a hybrid sensor system, including a plurality of fiber Bragg grating (FBG) sensors 118, such as those illustrated in FIG. 6, which may operate on a corresponding plurality of wavelengths, and a fiber-end sensor 216, which may be an acousto-optical sensor such as those described in detail above, operating at still another wavelength. Return optical signals from the sensors 118, 216 are demultiplexed by WDM 214, and returned to corresponding circulators 206, 212, which route the signals to corresponding first and second photodiodes (PDs) 218, 220 for signal processing. In FIG. 8A, the solid arrows represent optical paths and the broken line arrows indicate electrical signals.

FIG. 8B illustrates a distal end of a hybrid optical fiber sensor 222, which may be, as a non-limiting example, the hybrid optical fiber sensor discussed above with regard to FIG. 8A, including both FBG sensors 118 and the fiber-end sensor 216. The sensor portion in the non-limiting example of FIG. 8B is positioned adjacent the distal end 226 of an elongated guidewire body 224, although the arrangement is suitable to any interventional medical instrument. The distal end 226 may contain, or provide support for, the working element of instruments coaxially placed over the guidewire for, as non-limiting examples, stent graft deployment and/or sizing, balloon deployment, recanalization or crossing device, thrombectomy or other procedures, and may or may not require a tapered tip.

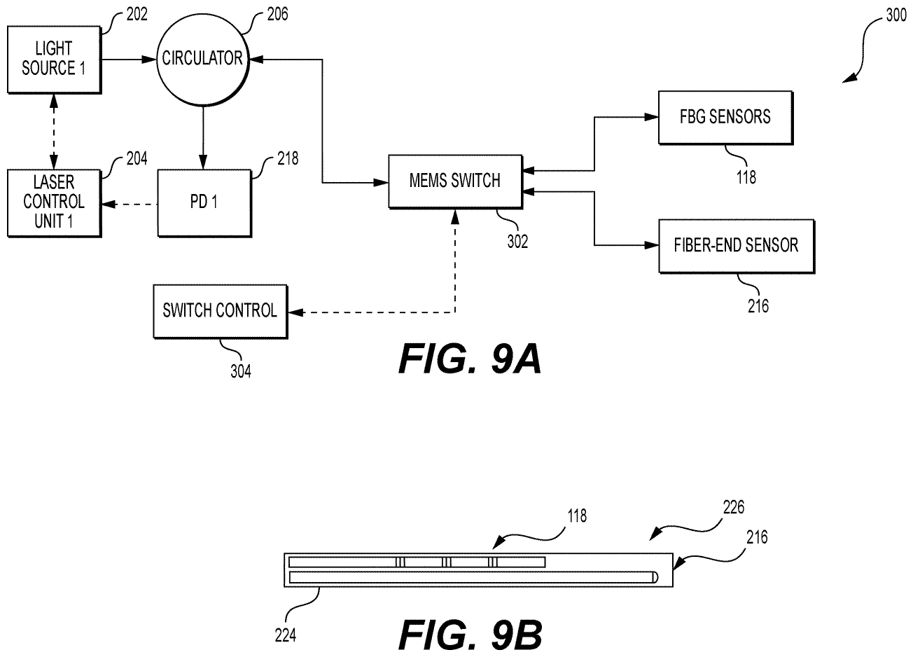

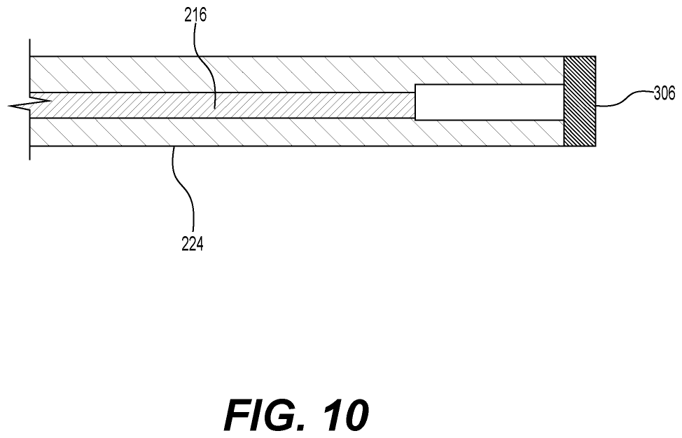

In the non-limiting example of FIGS. 8B, 9B, 12A and 12B, a number of fiber Bragg grating (FBG) sensors may be etched, or otherwise formed, in an optical fiber, with one further tip sensor (i.e., the fiber-end sensor) at the distal end of the guidewire. FIG. 9A diagrammatically illustrates an alternative system 300 for a hybrid optical fiber sensor, and FIG. 9B diagrammatically illustrates a distal end of the hybrid optical fiber sensor of FIG. 9A. In system 300, rather than having multiple light sources, only a single light source 202 is utilized, generating a beam which is received by a microelectromechanical systems (MEMS) switch (under control of switch controller 304), which allows for switching of signals between the FBG sensors 118 and the fiber-end sensor 216. As shown in the non-limiting example of FIG. 9B, the FBG sensors 188 and fiber-end sensor 216 under such a switching arrangement may easily be oriented within guidewire 224 separately from one another. FIG. 10 illustrates an alternative embodiment of the hybrid fiber sensor incorporating an extrinsic Fabry-Pérot interferometer (EFPI) 306 to replace the FBG sensors 118. This allows for making sensitive pressure measurements along with the acousto-optical sensing provided by the fiber-end sensor 216.

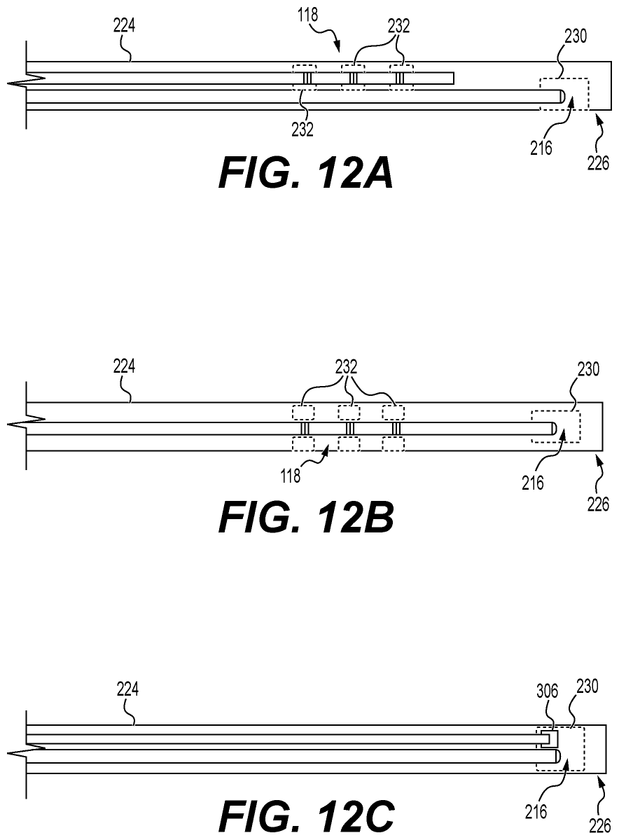

As shown in FIGS. 12A, 12B and 12C, the fiber-end sensor (FES) may be located within an acoustic window formed in the distal end of the guidewire. Each of FIGS. 12A, 12B and 12C illustrates a fiber-end sensor, such as fiber-end sensor 216 discussed above, mounted within an exemplary guidewire 224. As shown, in each of FIGS. 12A, 12B and 12C, the fiber-end sensor 216 is positioned within an acoustic window 230 formed in the distal end 226 of the guidewire 224. In FIG. 12A, the hybrid sensor may have the configuration of, for example, FIG. 9B, including FBG sensors 118 which, as shown, may each be located within respective temperature windows formed in the guidewire 224. FIG. 12B illustrates a similar sensor arrangement, but using the in-line hybrid configuration of FIG. 8B. FIG. 12C shows the guidewire 224 incorporating the hybrid sensor of FIG. 10, where the FBG sensors are replaced by the EPFI sensor 306.

The fiber-end sensor 118 offers tracking within an ultrasound image and B-mode (OnPoint) imaging from the fiber-end sensor location and, as shown, may be combined with other types of fiber sensors within the same guidewire 224. While one fiber end sensor is discussed above, it should be understood that there may be a series of resonant acousto-optic sensors in series proximate the fiber end or a relevant section of the medical device. In such arrangements, the resonant cavity walls are transparent, allowing different wavelengths to pass through adjacent acousto-optic sensors to reach the sensor configured for that wavelength. Such an arrangement allows for a distributed sensor array configuration to enable or enhance tracking, including multi-point tracking, trajectory forecasting and imaging modes, including color Doppler and shear wave. The acousto-optic sensors may be immediately adjacent to each other along the optical fiber or be spaced apart to accommodate the intended imaging environment and mode. The acousto-optic sensors may share the same waveguide or multiple waveguides and/or optical fibers. The temperature windows 232 for the FBG sensors 118 allow for additional temperature information to be gathered along with the tip tracking and/or OnPoint B-mode imaging. Such hybrid (or “multiple”) sensors may extract the optical signal information, such as intensity, spectral information, polarization status, etc., to provide accuracy of information (temperature and acoustic signal) better than single types of fiber sensors. It should be understood that the signal access windows for the fiber sensors may be further moved to a bending tip structure after mechanical optimization. It should be further understood that the temperature windows 232 are optional and are used if a more precise local temperature measuring point is required.

FIGS. 8B and 12B illustrate an alternative embodiment of the sensorized guidewire, in which the FBG sensors 118 and the fiber-end sensor 216 are integrated into the same optical fiber. In the further alternative embodiment of FIG. 12C, the additional FBG sensors 118 have been replaced with an extrinsic Fabry-Pérot interferometer (EFPI) 306, which allows for the collection of pressure information (as opposed to the collection of temperature information in the previous two embodiments). As shown, the EFPI 306 may be located in a common window 230 in the distal end 226 of the guidewire 224 with the fiber-end sensor 216. FIG. 13 illustrates an embodiment of 1.8 Fr sensorized catheter 60 incorporating a fiber-end sensor (FES) 62 and a transition region 64, which allows the coupling and decoupling of the fiber-end sensor 62 to the laser source, thereby permitting coaxial introduction of an instrument over the guidewire.

Further, it should be understood that the optical fiber may be received within a lumen or other types of tubular structures, for example, with the lumen or the like being in contact with, or formed integrally with, the elongated body. The optical sensors of the present invention are able to receive ultrasound transmissions from an externally positioned probe, internally positioned probe (e.g., transesophageal echocardiogram (TEE), intracardiac echocardiography (ICE), intravascular ultrasound (IVUS), endoscopic ultrasound (EUS), intraoperative probe, etc.) or an ultrasound patch. An example of a compact mixed ultrasound transducer, which may be used as such a probe, is shown in U.S. Patent Publication No. US 2025/0130322 A1, which is hereby incorporated by reference in its entirety. There is no requirement to integrate piezoelectric or transducer elements into the fiber itself, although such integration is herein contemplated. The sensorized medical instrument/device can be localized and tracked using 1) an external transducer (including an ultrasound patch); 2) a TEE or EUS probe; 3) an ICE catheter; 4) EUS; 5) interoperative transducer probe; or 6) piezoelectric (PZ) elements in fiber, parallel fibers, coaxial fibers, or a nearby sheath, catheter or wire with PZ elements.

As a result, additional diameter or proximity constraints related to integrated PZ elements are not absolutely required for the working guidewire configuration. Additionally, the on-tool optical based ultrasound transducers may be used to measure temperature; e.g., as described in U.S. Patent Publication No. US 2024/0358256 A1, which is hereby incorporated by reference, or to measure other physical, environmental or physiologic parameters.

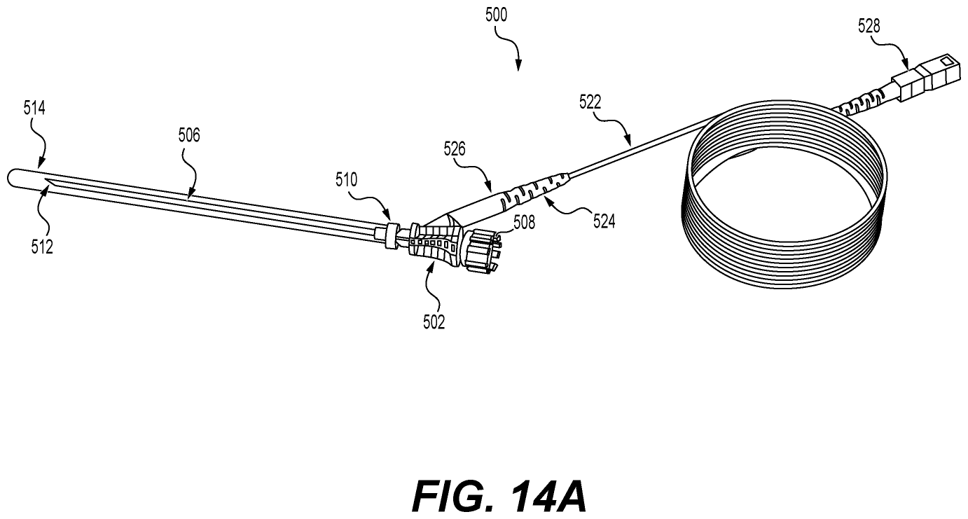

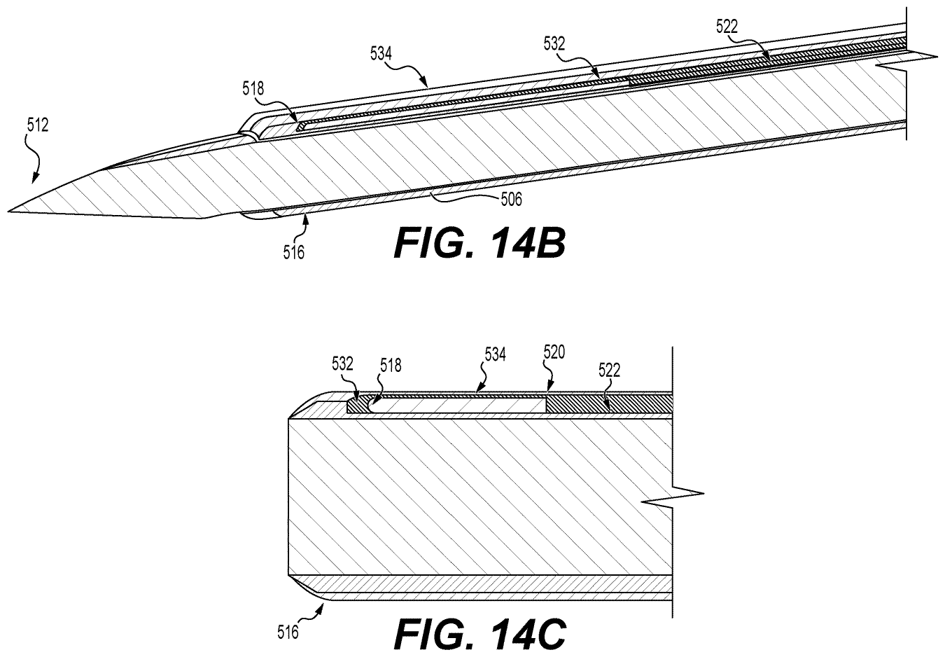

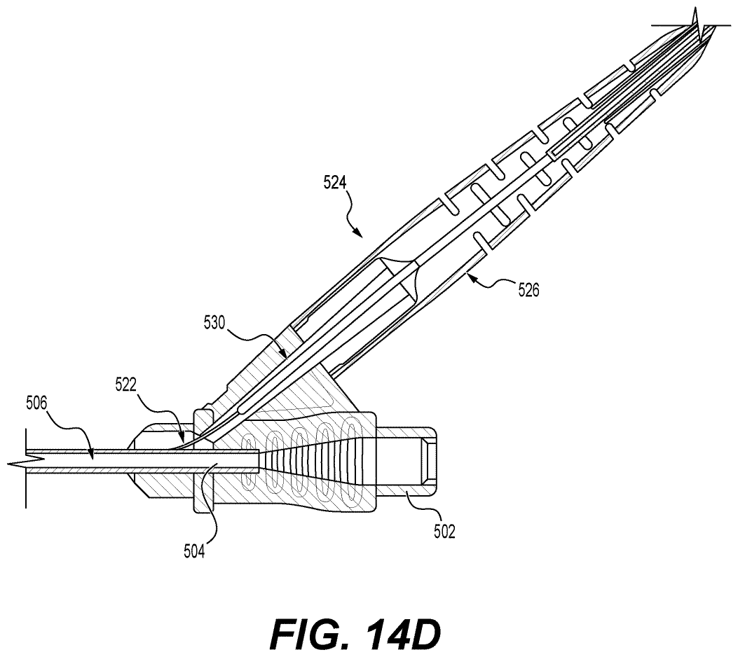

FIG. 14A illustrates an embodiment of a sensorized introducer 500 incorporating a fiber-end sensor. As shown in FIGS. 14A and 14B, as is conventionally known, the sensorized introducer 500 includes an introducer hub 502 for mounting of the proximal end 504 of an introducer tube 506. As is commonly known, as shown in FIG. 14A, the introducer hub 502 may include a passive trocar stylet hub 508 and a depth stopper 510. Extending from the introducer hub 502 is the introducer tube 506. In FIG. 14B, the introducer tube 506 is shown carrying an exemplary trocar stylet, which terminates at its distal end in a trocar stylet tip 512. In FIG. 14A, the introducer tube 506 is shown covered by a protective sheath 514 which, as is well-known, is typically provided for packaging purposes and is removed prior to use.

As best seen in FIG. 14B, the distal end 516 of the introducer tube 506 has a fiber-end sensor 518 mounted therein. As shown in FIG. 14C, a groove or channel 520 may be formed in the exterior surface of the introducer tube 506. Alternatively, the groove or channel may be formed within the tubular body of the introducer tube 506. The groove or channel 520 receives the fiber-end sensor 518 and the associated optical fiber 522. It should be understood that the fiber-end sensor 518 may be any of the sensors described in the previous embodiments. Further, it should be understood that the groove or channel 520 may have any suitable and/or desired dimensions. As a non-limiting example, the groove or channel 520 may be 100 μm deep and 300 μm wide.

As shown in FIGS. 14A and 14D, a fiber mount 524 (shown covered with a conventional strain relief cover 526) is affixed to the introducer hub 502 for carrying the proximal portion of the optical fiber 522, which may terminate in any suitable type of connector 528 for the optical fiber 522. The fiber mount 524 may include a fiber splice protector 530, as required. As shown, the proximal end of the optical fiber 522 extending from the strain relief 526 is jacketed (to provide protection from the environment).

The fiber-end sensor 518 and optical fiber 522 may be bonded to the distal end 516 of the introducer tube 506 using any suitable type of material, such as, but not limited to, an ultraviolet (UV) curable potting material 532. Further, the entire length of the introducer tube 506 may be coated with a protective coating 534, sealant or the like.

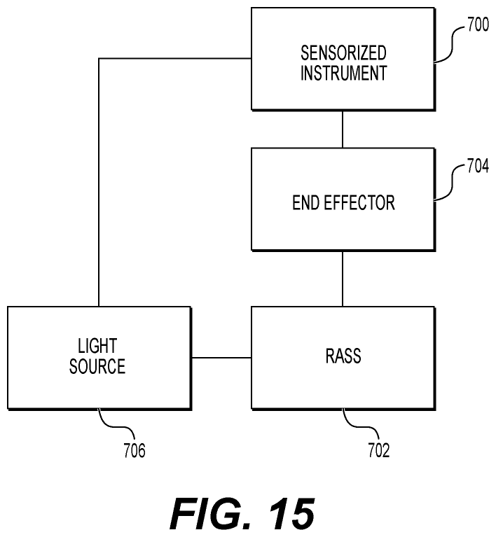

In the embodiment of FIG. 15, a sensorized instrument 700 is used with a robotic assisted surgical system (RASS) 702. The sensorized instrument 700 may be any of the sensorized instruments described herein. The light source for sensorized instrument 700 may be provided by the OSoC on the medical instrument hub that is selectively coupled to the robotic arm end effector 704 of RASS 702. The sensorized instrument 700 may have one or more optical sensors integrated in any of the ways described above. In this embodiment, the sensorized instrument's connection to the robotic arms must include a connection between the sensorized instrument's OSoC and the robotic system's imaging system, along with a processor for receiving the sensor-generated data and then determining sensor location, OnPoint Images and environmental or physiological parameters. The imaging system may be integrated with the robotic system 702 or be coupled to it. Such integration may also refine localization, where the acousto-optical sensor localization refines, augments, validates, or helps derive the positional information of the robotic end effector 704 with encoded position sensing joints or shape sensing positional or other localization technology. Either localization technology could be used to define failure modes or red flag identification of off-accurate data in order to reduce risk.

The robotic system 702 must also power the OSoC 706. Such cables can be routed conventionally through the robotic arms and to the end effectors 704, where the cables are then connected to the sensorized instrument hub for both powering the OSoC and transmitting the converted optical sensor data. Alternatively, the robotic system 702 may contain one or more lasers 706 which then connect to the instruments secured to the robotic working components (i.e., end effectors 704) and further provides a connection to the robotic system's imaging system. Such lasers may be placed nearer to the distal end of the arm to minimize optical fiber routing. The routing of the optical fibers from the laser 706 must take into consideration the articulation and bending of the robotic arms. Suitable optical fibers can be selected with a bending curvature that will not affect the integrity of the wavelengths within the waveguide. Non-limiting examples of such optical cables include polarization-maintaining fibers, reduced cladding fibers and bend-loss insensitive fibers. These optical fibers may be contained within a flexible cable routed alongside the articulated arms, either internally or externally. Appropriate anchor points may be included to enable the fiber optical cable to follow the articulation of the robotic arms without undue bending that affects the wavelengths within the optical cable. Alternatively, the laser 706 may be positioned at the end effector 704, or as close as possible to minimize the optical cable length. Thus, the optical signal is converted to its electrical signal adjacent the sensorized instrument 700, and corresponding electrical wires for carrying the converted signal may be conventionally routed along the other robotic arms to the processor for image generation, instrument tracking and output of environmental or physiological data.

As another non-limiting example, the sensorized instrument may be removably coupled to a robotic end effector or integrated into a robotic end effector (e.g., as part of, or in use with, a robotic grasper). When the sensor is located at the end of the integrated instrument (which may be removeable), the robotic end effector may not be a tool mounted on the robotic arm, for example, but, rather, a grasper that directly manipulates tissue. Thus, in this non-limiting example, the grasper may have an integrated sensor at the distal end, secured via an exterior channel, an interior lumen or reinforced soft cabling/tubing. It should be understood that any necessary articulation for the optical fiber may be used along the robotic arm a laser is used, as opposed to an Optical System on a Chip (OSoC).

It is to be understood that the medical devices with integrated ultrasound optical sensors are not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A medical device with an integrated ultrasound optical sensor, comprising:
a device proximal end;
a body extending from the device proximal end;
a device distal end;
a working section on the body;
at least one ultrasound optical sensor secured to the body proximate the distal end;
a light source configured to provide light to the optical ultrasound sensor via the optical fiber;
a photodetector configured to receive an optical signal from the optical ultrasound sensor; and
a controller configured to:
control the light source;
receive optical data based on the optical signal from the photodetector; and
perform at least one of image generation and location determination based on the optical data.

2. The medical device with an integrated ultrasound optical sensor as recited in claim 1, wherein the at least one ultrasound optical sensor comprises a sensor portion and an optical fiber in optical communication therewith, the sensor portion being positioned adjacent a distal end of the body.

3. The medical device with an integrated ultrasound optical sensor as recited in claim 1, wherein the controller is further configured to perform physiological analysis based on the optical data.

4. The medical device with an integrated ultrasound optical sensor as recited in claim 3, wherein the physiological analysis determines one or more of temperature, pressure or nerve activity parameters.

5. The medical device with an integrated ultrasound optical sensor as recited in claim 2, further comprising:
at least one optical sensor for tracking and/or imaging; and
at least one optical sensor for physiological sensing.

6. The medical device with an integrated ultrasound optical sensor as recited in claim 1, further comprising a second ultrasound optical sensor for tracking, imaging and/or physiologic sensing.

7. The medical device with an integrated ultrasound optical sensor as recited in claim 1, wherein the working section is selected from the group consisting of an ablation element, a delivery component, a grasping tool, a surgical tool end, a biopsy tool, a monitoring instrument, and a coagulation module.

8. The medical device with an integrated ultrasound optical sensor as recited in claim 7, wherein the at least one ultrasound optical sensor is received within an inner lumen or an exterior channel on the medical device.

9. The medical device with an integrated ultrasound optical sensor as recited in claim 1, further comprising a chip-based laser secured to the device proximal end, the at least one ultrasound sensor being in communication with the chip-based laser.

10. The medical device with an integrated ultrasound optical sensor as recited in claim 2, wherein the body is hollow and the at least one ultrasound optical sensor is secured against the body.

11. The medical device with an integrated ultrasound optical sensor as recited in claim 10, wherein the body is covered with a cover layer, and wherein the optical fiber is embedded therein.

12. The medical device with an integrated ultrasound optical sensor as recited in claim 11, wherein an annular layer is positioned adjacent the cover layer, the sensor portion being embedded in the annular layer, and wherein the annular layer is formed from an acoustic matching material.

13. The medical device with an integrated ultrasound optical sensor as recited in claim 1, wherein the at least one ultrasound optical sensor is an acousto-optic sensor.

14. The medical device with an integrated ultrasound optical sensor as recited in claim 1, wherein the medical device is in communication with a tunable light source.

15. The medical device with an integrated ultrasound optical sensor as recited in claim 1, wherein the device proximal end comprises a proximal hub.

16. The medical device with an integrated ultrasound optical sensor as recited in claim 1, wherein the at least one ultrasound optical sensor is calibrated to provide multimodality data.

17. The medical device with an integrated ultrasound optical sensor as recited in claim 16, wherein the controller is further configured to:
receive the multimodality data from the at least one ultrasound optical sensor;
process the multimodality data using a machine learning model; and
generate at least one output including an ultrasound-centered model for determining a finite analysis of tissue sensed by the at least one ultrasound optical sensor to determine Pennes Bioheat equation effects upon the tissue, an indication of a cumulative lethal tissue zone, an ultrasound-centered model with spatial coordinates for interstitial pressure, or co-register point-based pressure measurement spatial data to match sensor data with a MRI DWI/ADC map.

18. A working guidewire with an integrated ultrasound optical sensor, comprising:
a guidewire having opposed distal and proximal ends, an acoustic window being formed in the distal end;
an acousto-optic sensor received within the guidewire, with a fiber-end sensor portion thereof positioned within the acoustic window.

19. The working guidewire with an integrated ultrasound optical sensor as recited in claim 18, wherein an additional sensor is received within the guidewire.

20. The working guidewire with an integrated ultrasound optical sensor as recited in claim 19, wherein the additional sensor comprises an optical fiber with at least one fiber Bragg grating.

21. The working guidewire with an integrated ultrasound optical sensor as recited in claim 19, wherein the at additional sensor comprises an extrinsic Fabry-Perot interferometer.

22. The working guidewire with an integrated ultrasound optical sensor as recited in claim 18, further comprising:
a light source configured to provide light to the acousto-optic sensor via an optical fiber;
a photodetector configured to receive an optical signal from the acousto-optic sensor; and
a controller configured to:
control the light source;
receive optical data based on the optical signal from the photodetector; and
perform at least one of image generation and location determination based on the optical data.

23. The working guidewire with an integrated ultrasound optical sensor as recited in claim 18, further comprising at least two or more acousto-optic sensors for generation of Doppler or shear wave images.

24. A sensorized introducer, comprising:
an introducer tube having opposed distal and proximal ends, a surface of the introducer tube having a groove formed therein;
an introducer hub, the proximal end of the introducer being received by the introducer hub; and
an acousto-optic sensor received within the groove, with a fiber-end sensor portion thereof positioned adjacent the distal end of the introducer tube, a proximal portion of an optical fiber of the acousto-optic sensor extending through the introducer hub.

25. The sensorized introducer as recited in claim 24, wherein the fiber-end sensor portion and at least a portion of the optical fiber are bonded within the groove.

26. The sensorized introducer as recited in claim 24, wherein the introducer tube is coated with a protective coating.

27. The sensorized introducer as recited in claim 24, further comprising a fiber mount mounted on the introducer hub for carrying at least a portion of the optical fiber.

28. The sensorized introducer as recited in claim 24, further comprising:
a light source configured to provide light to the acousto-optic sensor via the optical fiber;
a photodetector configured to receive an optical signal from the acousto-optic sensor; and
a controller configured to:
control the light source;
receive optical data based on the optical signal from the photodetector; and
perform at least one of image generation and location determination based on the optical data.

29. A robotic assisted surgical system, comprising:
at least one end effector; and
the medical device with an integrated ultrasound optical sensor as recited in claim 1, the medical device with an integrated ultrasound optical sensor being coupled to the at least one end effector.
